(19) United States
(12) Patent Application Publication
(10) Pub. No.: US 2002/0087216 A1
(43) Pub. Date: Jul. 4, 2002

(54) Prosthetic walking system

(76) Inventors: Stewart L. Atkinson, Bainbridge Island, WA (US); Donald L. Poggi, Bainbridge Island, WA (US); Jeremy Adelson, Bainbridge Island, WA (US)

Correspondence Address: MICHAEL BEST & FRIEDRICH, LLP, 100 E WISCONSIN AVENUE, MILWAUKEE, WI, 53202, US

(21) Appl. No.: 10/082,678

(22) Filed: Feb. 25, 2002

Related U.S. Application Data
(63) Continuation-in-part of application No. 09/369,206, filed on Aug. 5, 1999, now Pat. No. 6,350,286.
Application No. 09/369,206 is a continuation of application No. 08/903,529, filed on Jul. 30, 1997, now abandoned.
Application No. 08/903,529 is a continuation of application No. 08/602,241, filed on Feb. 16, 1996, now Pat. No. 5,800,568.

(51) Int. Cl. A61F 2/66

(52) U.S. Cl. 623/38; 623/52

(57) ABSTRACT

A prosthetic walking system including a pylon, a prosthetic ankle, and a prosthetic foot, any two or more of which can be integrally connected into a single, continuous unit. The prosthetic ankle can be a rearwardly-facing, generally C-shaped member, and may include an upper leg, an interconnecting portion, and a lower leg. The prosthetic ankle may include a weakened portion so that the prosthetic walking system is better adapted to flex at the prosthetic ankle rather than at the pylon. A link or link assembly can be coupled to at least one of the pylon and the upper leg of the prosthetic ankle and to at least one of the lower leg of the prosthetic ankle and the prosthetic foot in order to limit the displacement between the upper leg and the lower leg of the prosthetic ankle.

### RELATED APPLICATIONS

This application is a continuation-in-part of U.S. patent application Ser. No. 09/369,206, filed Aug. 5, 1999, issued ______ as U.S. patent No. ______ , which is a continuation of U.S. patent application Ser. No. 08/903,529, filed Jul. 30, 1997, which is a continuation of U.S. patent application Ser. No. 08/602,241, filed Feb. 16, 1996, issued as U.S. patent No. 5,800,568 on Sep. 1, 1998.

### FIELD OF THE INVENTION

This invention relates generally to prosthetic devices, and more particularly to an apparatus and method for a prosthetic walking system including a pylon, a prosthetic ankle, and a prosthetic foot.

### BACKGROUND OF THE INVENTION

Prosthetic walking systems to help leg amputees regain significant walking capability. Many conventional prosthetic walking systems, such as the walking system A shown in FIG. 1, include a pylon B rigidly connected to a prosthetic foot C having a heel portion D, a flat bottom portion F, and a toe portion T. Amputees often experience instability while using the walking system A because the flat bottom portion F of the prosthetic foot C does not contact the ground quickly enough after the heel portion D contacts the ground (i.e., at “heel-strike”). Before the flat bottom portion F of the prosthetic foot C contacts the ground, an amputee's weight W is largely supported by the heel portion D of the prosthetic foot C. The flat bottom portion F of the prosthetic foot C does not contact the ground during the amputee's gait until just prior to the amputee's weight W coming off of the toe portion T (i.e., at “toe-off”). Amputees also experience instability while using the walking system A because the walking system A does not include any cushioning between the pylon B and the prosthetic foot C.

Many conventional prosthetic walking systems, such as the walking system S shown in FIG. 2, also include a resilient ankle E positioned between the pylon B and the prosthetic foot C to help alleviate the instability of the walking system A. However, amputees still experience instability while using the walking system S because the amputee's weight W at heel-strike causes the toe portion T of the prosthetic foot C to pivot upward toward the pylon B rather than downward toward the ground. As a result, the flat bottom portion F of the prosthetic foot C does not contact the ground for an even longer portion of the amputee's gait than with the walking system A.

The resilient ankle E provides cushioning by biasing the pylon B and the prosthetic foot C apart from one another. However, the resilient ankle E cannot be biased to provide too much cushioning, or the pylon B becomes displaced too far from the prosthetic foot C. If the pylon B is displaced too far from the prosthetic foot C, the pylon B is forced out of a desired vertical alignment and the prosthetic foot C is forced out of a desired horizontal alignment.

Many conventional prosthetic walking systems employ separate components for the pylon B, the resilient ankle E, and the prosthetic foot C. These prosthetic walking systems require the manufacture and assembly of several separate components. Connecting several components often results in instabilities between the components and can significantly increase manufacturing and assembly costs of the system. For example, the connection between the pylon B and the resilient ankle E may become weakened or loosened, causing instability for the amputee.

In many conventional prosthetic walking systems, the resilient ankle E provides cushioning by flexing when an amputee's weight is placed on the prosthetic walking system. However, forces exerted upon the pylon B during flexure of the resilient ankle E can place undesirable and damaging stresses on the pylon B.

In light of the problems and limitations described above, a need exists for a method and apparatus for a prosthetic walking system having improved comfort, motion, and stability. Specifically, a need exists for a prosthetic walking system that places a toe portion of a prosthetic foot into contact with the ground more quickly after a heel portion of the prosthetic foot contacts the ground during an amputee's gate. A need also exists for a prosthetic walking system including a resilient ankle that provides adequate cushioning between a pylon and a prosthetic foot while limiting the displacement between the pylon and the prosthetic foot. In addition, a need exists for a resilient ankle that can be adjusted to define a maximum displacement between a pylon and a prosthetic foot. A need further exists for a prosthetic walking system including a pylon, a resilient ankle, and a prosthetic foot, or any combination thereof, comprised of a single, integral unit. Furthermore, a need exists for a prosthetic walking system designed to flex under an amputee's weight at a resilient ankle, rather than at a pylon connected thereto. Finally, a need exists for a prosthetic walking system that is comprised of inexpensive components, that is simple to manufacture, that is easy to assemble, and that is easy to repair. Each embodiment of the present invention achieves one or more of these results.

### SUMMARY OF THE INVENTION

Some highly preferred embodiments of the present invention provide a prosthetic walking system including a pylon having an upper end for attachment to an amputee's leg stump and a lower end coupled to a prosthetic ankle and a heel portion of a prosthetic foot. The prosthetic ankle can include an upper leg coupled to the lower end of the pylon, a lower leg coupled to the heel portion of the prosthetic foot, and an interconnecting portion coupled between the upper leg and the lower leg. In some embodiments, the upper leg and the lower leg are substantially straight and the interconnecting portion is substantially arcuate. In other embodiments, the upper leg, the lower leg, and the interconnecting portion are each substantially arcuate. In some embodiments, the upper leg is shorter than the lower leg. Also, the upper leg may include one or more apertures or connections adapted to receive the pylon in at least two different positions. In some embodiments, the interconnecting portion of the prosthetic ankle is coupled between an anterior portion of the upper leg and an anterior portion of the lower leg. In these embodiments, the prosthetic ankle is preferably a rearwardly-facing, generally C-shaped member.

The pylon, the prosthetic ankle, and the prosthetic foot can each be separate components that are assembled to form the prosthetic walking system. Alternatively, the pylon, the prosthetic ankle, and the prosthetic foot, or any combination thereof, can be integrally connected, i.e., formed together into a continuous, integral unit. For example, the pylon and the prosthetic ankle can be integrally connected and can be coupled to a separate prosthetic foot. Similarly, the prosthetic ankle and the prosthetic foot can be integrally connected and can be coupled to a separate pylon.

The prosthetic ankle can include one or more weakened portions that are less resistant to bending than one or more strengthened portions that are more resistant to bending in order to define where the prosthetic ankle will flex when a load is placed on the prosthetic ankle. For example, the prosthetic ankle can include one or more weakened portions having a first cross-sectional area that is smaller than one or more strengthened portions having a second cross-sectional area. In order to define the weakened and strengthened portions of the prosthetic ankle, the width of either the upper leg or the interconnecting portion of the prosthetic ankle is preferably smaller than the width of the pylon, so that the prosthetic walking system flexes under an amputee's weight at the prosthetic ankle rather than at the pylon. The weakened portion of the prosthetic ankle can be positioned asymmetrically with respect to a longitudinal axis of the pylon according to whether the prosthetic walking system is designed for attachment to the amputee's left or right side. The asymmetrically-positioned, weakened portion also allows the prosthetic ankle to flex inwardly (i.e., medially) or outwardly (i.e., laterally) with respect to a longitudinal midline that divides the amputee's body in half. Also, the cross-sectional shape of the prosthetic ankle can have a lower moment of inertia than the cross-sectional shape of the pylon, or can otherwise be stiffer than the prosthetic ankle, so that the prosthetic ankle flexes before the pylon flexes when an amputee's weight is placed on the prosthetic walking system. In some embodiments, the pylon has a substantially circular cross-sectional shape, while the prosthetic ankle has a substantially rectangular cross-sectional shape. The pylon is preferably constructed of a relatively light-weight and resilient material such as carbon composite, while the prosthetic ankle is preferably constructed of a relatively strong and flexible material such as fiberglass.

The prosthetic foot includes a toe portion that preferably has two or more toe sections (i.e., a split keel). The toe sections, if employed, can be of any relative size, any shape, and can be in any position on the toe portion of the prosthetic foot or in any position with respect to the remainder of the prosthetic walking system. For example, one of the toe sections can be smaller than the other toe section or sections and can be positioned medially with respect to a longitudinal axis of the pylon. The smaller, medially-positioned toe section preferably provides a preference toward where the amputee's big toe would be located on the amputee's left or right side.

Preferably, at least one link is provided to limit the displacement between the pylon and the prosthetic foot when the amputee's weight is not loading the prosthetic ankle. In order to limit the displacement between the pylon and the prosthetic foot, at least one link can be provided to limit the displacement between the upper leg and the lower leg of the prosthetic ankle. In some embodiments, at least one link is coupled between at least one of the pylon and the upper leg of the prosthetic ankle and at least one of the lower leg of the prosthetic ankle and the prosthetic foot. In some embodiments, a link assembly, including one or more links and the components that secure the link or links to the prosthetic walking system, is used to limit the displacement between the pylon and the prosthetic foot. The link assembly can be coupled between at least one of the pylon and the upper leg of the prosthetic ankle and at least one of the lower leg of the prosthetic ankle and the prosthetic foot. For example, in the case of a rearwardly-facing, C-shaped ankle as described above, link assembly can be coupled between a posterior portion of the upper leg and a posterior portion of the lower leg of the prosthetic ankle. For each possible configuration, the link assembly preferably defines a maximum displacement between the upper leg and the lower leg of the prosthetic ankle.

In some embodiments, the link comprises a strap having a top portion coupled between the pylon and the upper leg of the prosthetic ankle, a bottom portion coupled between the prosthetic ankle and the heel portion of the prosthetic foot, and an intermediate portion coupled between the top portion and the bottom portion. The length of the intermediate portion can define the maximum displacement between the upper leg and the lower leg of the prosthetic ankle.

The link can also be comprised of a resilient belt or band having an upper portion coupled to at least one of the pylon and the upper leg of the prosthetic ankle and a lower portion coupled to at least one of the lower leg of the prosthetic ankle and the prosthetic foot. In some embodiments, the resilient belt is a cord having two or more lengths in order to distribute the biasing force over the two or more lengths. In these embodiments, a middle portion of the cord can form the upper portion of the resilient belt, and the ends of the cord can form the lower portion of the resilient belt, or vice versa.

Some embodiments include a link assembly comprised of a first link, a second link, and a heel. Preferably, a first portion or end of the first link is coupled to at least one of the pylon and the upper leg of the prosthetic ankle, a second portion or end of the first link is coupled to a first portion or end of the second link, and a second portion or end of the second link is coupled to the heel. The heel is coupled to at least one of the lower leg of the prosthetic ankle and the prosthetic foot. An adjustment screw is preferably coupled between either the first link or the second link and either the pylon, the upper leg of the prosthetic ankle, or the heel. The adjustment screw can be adjusted in order to move the link assembly and to vary the maximum displacement between the upper leg and the lower leg of the prosthetic ankle.

The link can also comprise a hydraulic or pneumatic cylinder coupled to at least one of the pylon and the upper leg of the prosthetic ankle and to at least one of the lower leg of the prosthetic ankle and the prosthetic foot. Preferably, the pressure within and the initial position of the hydraulic or pneumatic cylinder can be adjusted in order to vary the maximum displacement between the upper leg and the lower leg of the prosthetic ankle.

According to a method of the invention, a prosthetic walking system is attached to an amputee. The prosthetic walking system includes a pylon, a prosthetic ankle, and a prosthetic foot. The prosthetic ankle preferably includes an upper leg, a lower leg, and an interconnecting portion. A link or link assembly can be provided to limit the displacement between the upper leg and the lower leg. Preferably, a maximum displacement between the upper leg and the lower leg is limited with the link or link assembly. Also preferably, the link or link assembly can be adjusted to change the defined maximum displacement, such as by an adjustment screw. In other embodiments, a pressure in a hydraulic or pneumatic cylinder can be changed to adjust the link or link assembly.

Further objects and advantages of the present invention, together with the organization and manner of operation thereof, will become apparent from the following detailed description of the invention when taken in conjunction with the accompanying drawings, wherein like elements have like numerals throughout the drawings.

### BRIEF DESCRIPTION OF THE DRAWINGS

The present invention is further described with reference to the accompanying drawings, which show a preferred embodiment of the present invention. However, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a side elevational view of a prior art walking system;

FIG. 2 is a side elevational view of another prior art walking system;

FIG. 3 is an exploded perspective view of a prosthetic ankle for use with a prosthetic walking system of the present invention;

FIG. 4 is top plan view of the prosthetic ankle of FIG. 3;

FIGS. 5A and 5B are side elevational views of the prosthetic walking system including the prosthetic ankle of FIG. 3;

FIGS. 6A, 6B, and 6C are front and side elevational views of the prosthetic walking system including the prosthetic ankle of FIG. 3;

FIGS. 7A and 7B are top plan views of alternative embodiments of the prosthetic ankle of FIG. 3;

FIG. 8 is a side elevational view of an alternative embodiment of the prosthetic walking system of the present invention;

FIGS. 9A, 9B, 9C, and 9D are front perspective, front elevational, side elevational, and rear elevational views, respectively, of an alternative embodiment of the prosthetic walking system of the present invention;

FIGS. 10A and 10B are exploded side elevational and side elevational views, respectively, of an alternative embodiment of the prosthetic walking system of the present invention;

FIGS. 11 is a perspective view of another alternative embodiment of the prosthetic walking system of the present invention;

FIG. 12 is a rear elevational view of the prosthetic walking system of FIG. 11;

FIG. 13 is an exploded rear perspective view of the prosthetic walking system of FIG. I1;

FIG. 14 is an exploded front perspective view of another alternative embodiment of the prosthetic walking system of the present invention, including an adjustable linkage assembly;

FIG. 15 is a front perspective view of the adjustable linkage assembly of FIG. 14;

FIG. 16 is a side elevational view of the adjustable linkage assembly of FIG. 14;

FIG. 17 is a partial side elevational view of the prosthetic walking system of FIG. 14;

FIG. 18 is a partial rear elevational view of the prosthetic walking system of FIG. 14, taken along line 18-18 of FIG. 17;

FIG. 19 is a rear perspective view of the prosthetic walking system of FIG. 14; and

FIG. 20 is a rear perspective view of another alternative embodiment of the prosthetic walking system of the present invention.

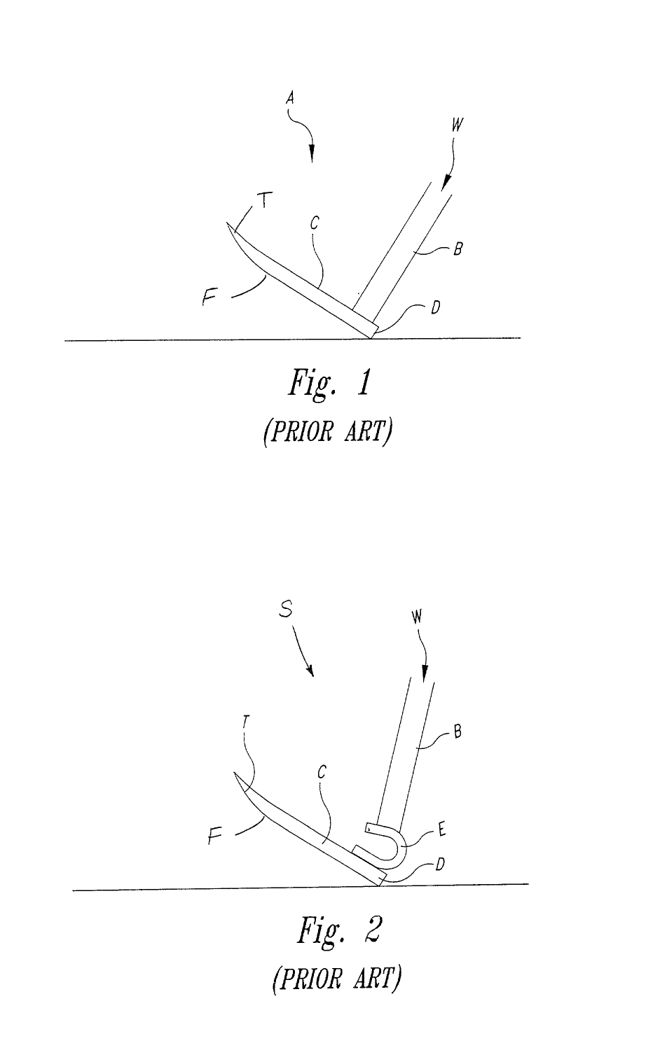

### DETAILED DESCRIPTION OF THE PREFERRED EMBODIMENTS

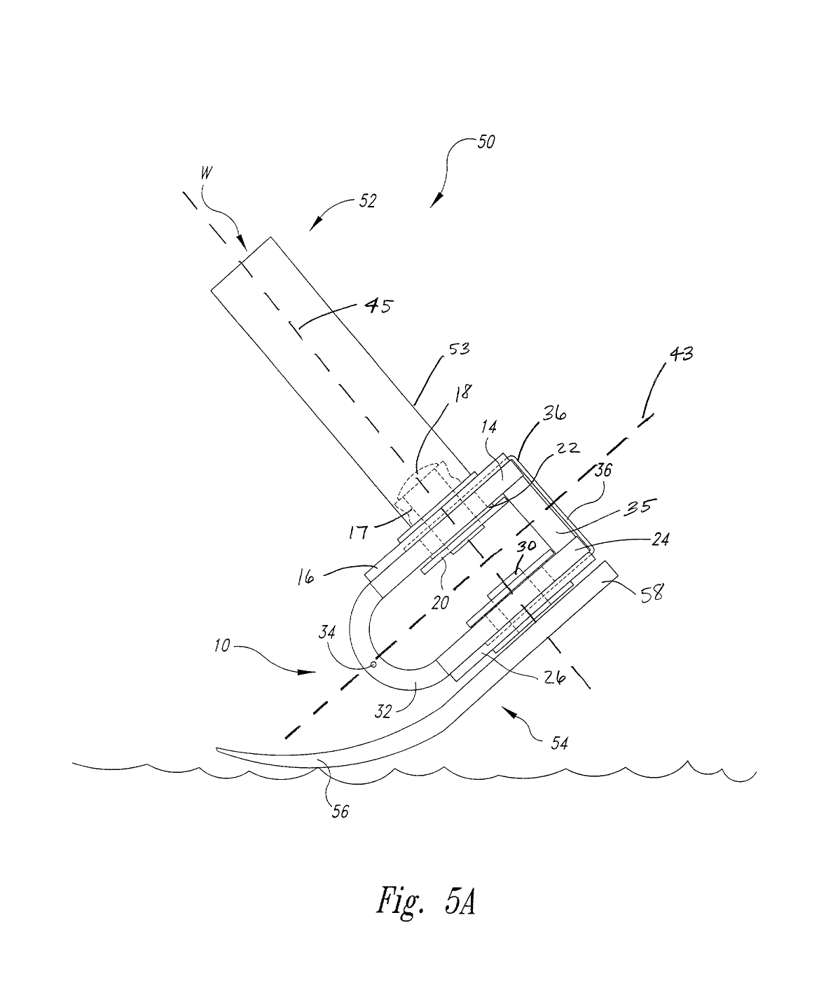

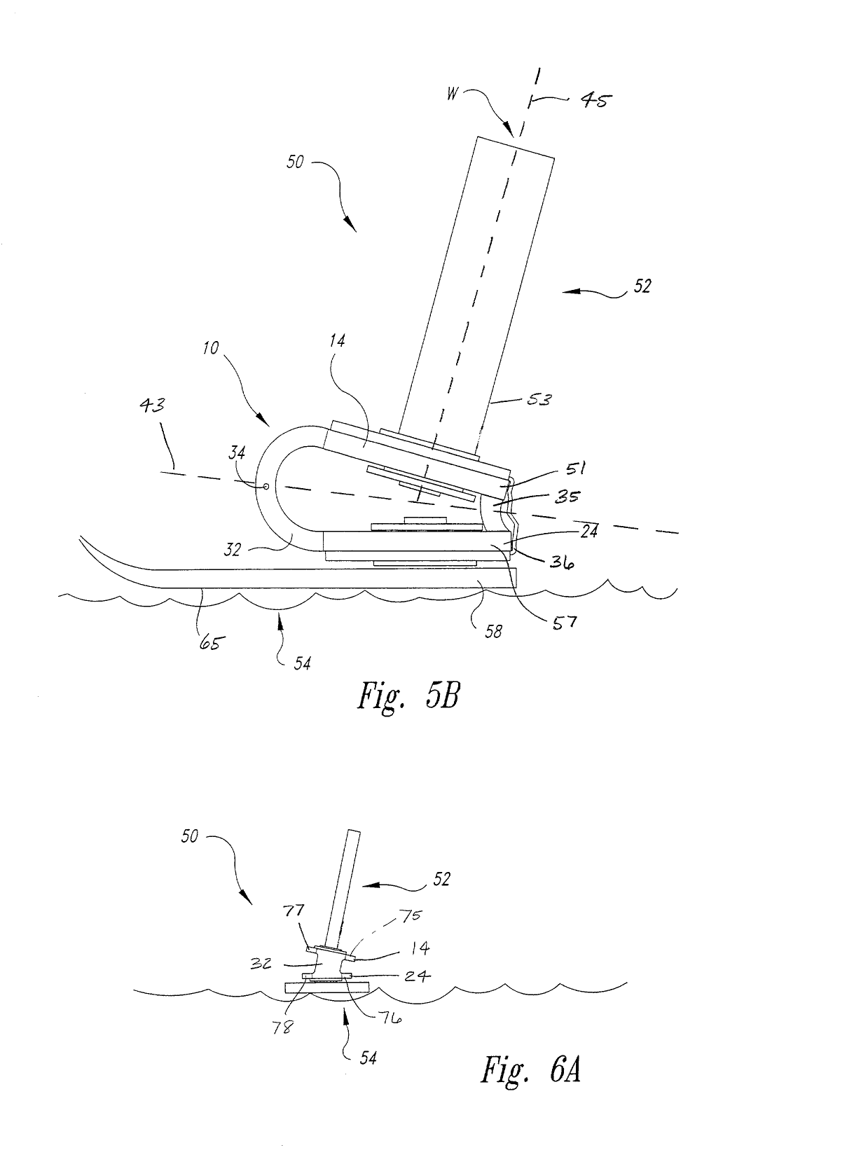

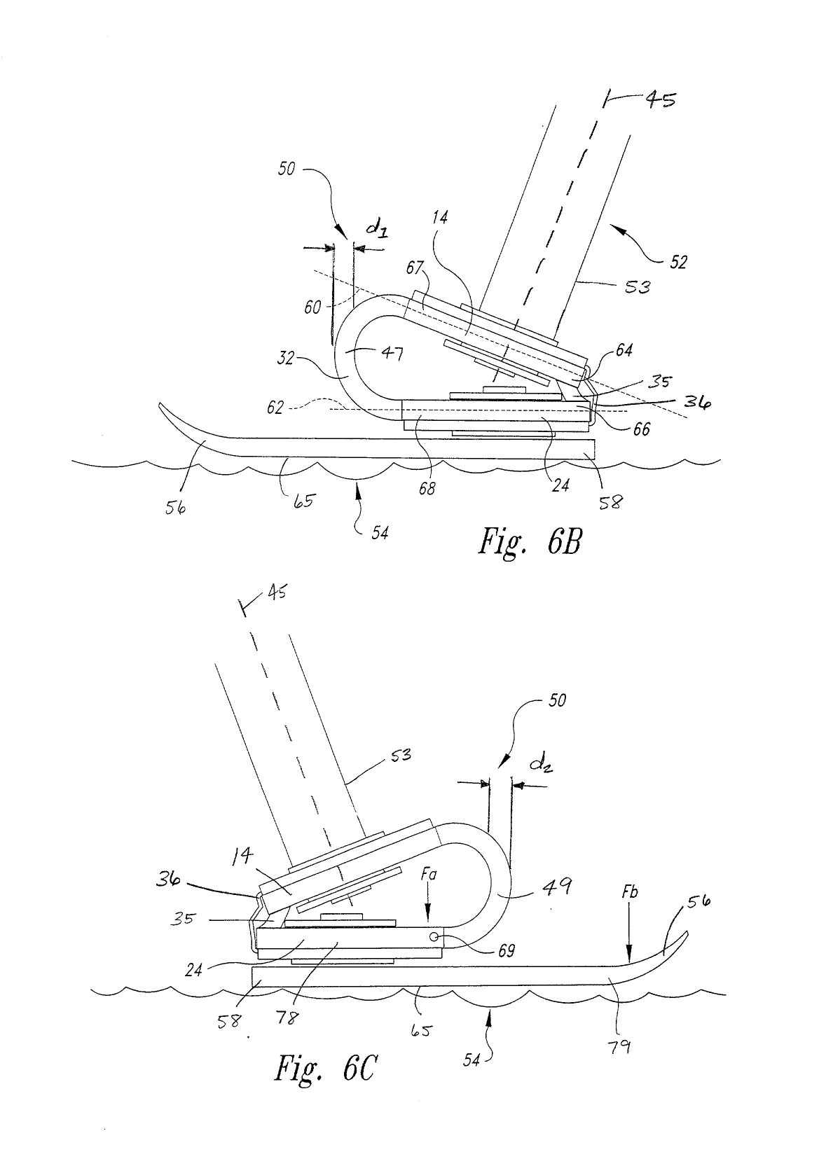

FIGS. 5A-6C illustrate one embodiment of a prosthetic walking system 50 according to the present invention. The prosthetic walking system 50 includes a pylon 52 that is securable to an amputee's leg stump, a prosthetic ankle assembly 10 coupled to a lower end 53 of the pylon 52, and a prosthetic foot 54 coupled to the prosthetic ankle assembly 10.

An upper end (not shown) of the pylon 52 is securable to an amputee's leg stump and serves as a portion of the amputee's leg. The pylon 52 can be coupled to the amputee's leg stump in any conventional manner, such as by coupling the upper end of the pylon 52 to an amputation socket (not shown) that is securable to the amputee's stump. The pylon 52 is preferably removably coupled to the amputation socket so that the prosthetic walking system 50 can be easily removed and replaced with a prosthesis having different properties when desired by the amputee. The pylon 52 can be of any suitable length for the length of the amputee's leg stump. For example, the upper end of the pylon 52 may terminate below or at the amputee's knee, within the length of the amputee's thigh, or near the amputee's hip. Moreover, the pylon 52 may also be attached to a prosthetic knee joint to form a dual-joint prosthetic walking system. The pylon 52 can be constructed of any suitable, weight-bearing material, such as titanium, carbon-fiber composite, fiberglass, plastic, aluminum, steel, or other metals or metal alloys, hardened polymers, and the like. The pylon 52 can have any suitable cross-sectional shape, such as a circular, oval, triangular, rectangular, pentagonal, octagonal, or irregular-shaped cross-sections. In addition, the pylon 52 can be constructed of more than one member. For example, the pylon 52 can be constructed of a first longitudinal member that simulates the fibula and a second longitudinal member that simulates the tibia.

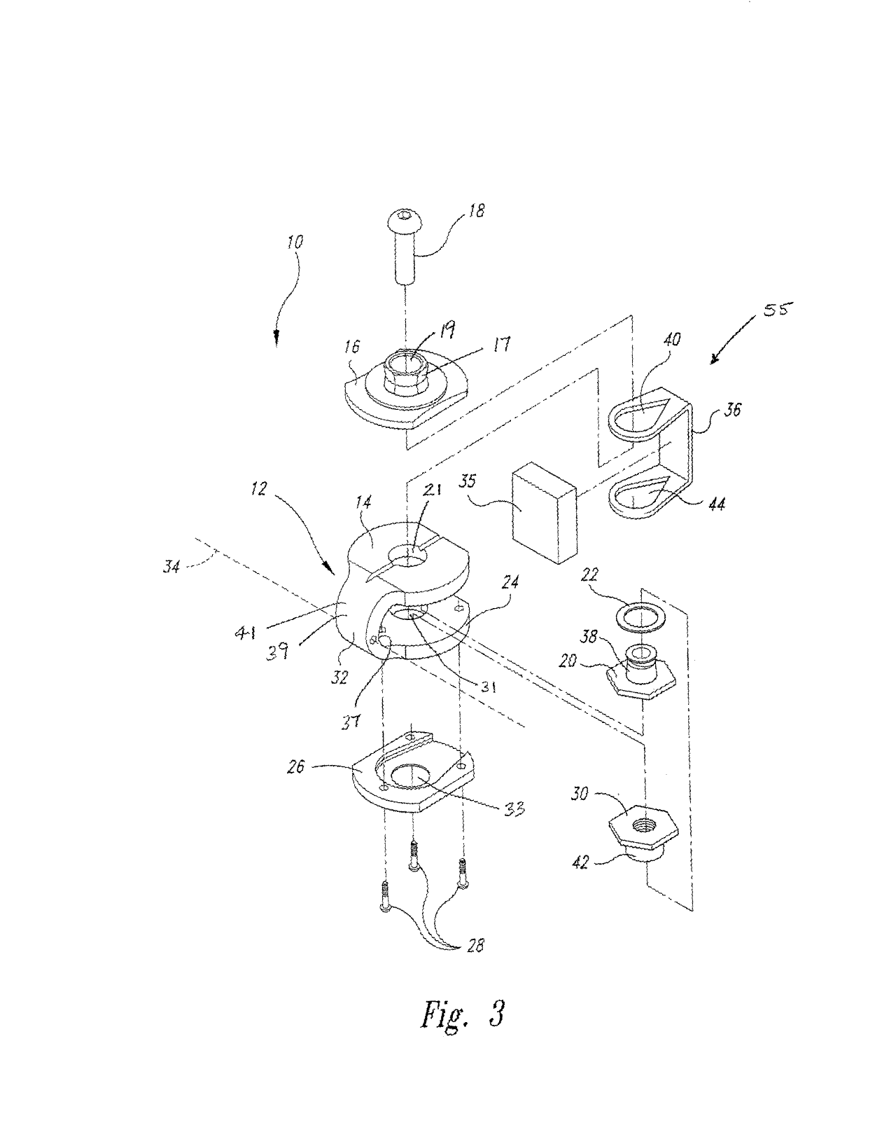

FIG. 3 illustrates one embodiment of the prosthetic ankle assembly 10 for use in the prosthetic walking system 50 shown in FIGS. 5A-6C. The prosthetic ankle assembly 10 can be used with any desired pylon and prosthetic foot, and is shown and described for use with the prosthetic walking system 50 by way of example only. The prosthetic ankle assembly 10 includes a prosthetic ankle 12 preferably having a C-shape. Preferably, the opening of the C-shape of the prosthetic ankle 12 faces rearward (i.e., toward the posterior direction). However, in other embodiments, the opening of the C-shape of the prosthetic ankle 12 may face forward (i.e., toward the anterior direction).

The prosthetic ankle 12 preferably includes an upper leg 14, a lower leg 24, and an interconnecting portion 32. The upper leg 14, the lower leg 24, and the interconnecting portion 32 can each have any shape suitable for defining a prosthetic ankle 12 having a top portion, a bottom portion, and an interconnecting portion. In some embodiments, such as that shown in FIGS. 5A, 5B, 6B, and 6C, the upper leg 14 and the lower leg 24 are substantially straight, while the interconnecting portion 32 is substantially arcuate. However, the upper leg 14, the lower leg 24, and the interconnecting portion 32 can each be substantially arcuate.

The prosthetic ankle 12 is preferably constructed of one or more materials that are relatively strong, stiff, resilient, flexible, dimensionally stable, fatigue-resistant, abrasion-resistant, wear-resistant, impact-resistant, and have a low coefficient of friction. In some embodiments, the prosthetic ankle 12 is constructed of a material that is more compliant (i.e., less resistant to bending) than the material used to construct the pylon 52. For example, the prosthetic ankle 12 can be constructed of fiberglass, while the pylon 52 is constructed of carbon-fiber composite. In other embodiments, other materials can be used for the construction of the prosthetic ankle 12, either alone or in combination, such as DELRIN® (E. I. du Pont de Nemours and Company) or its generic equivalent Acetal, Ertalyte® (Quadrant DSM Polymer Corporation), Noryl® (General Electric Company), UHMW Polyethylene or one of its commercial versions Lenite® (Westlake Plastics Company) or Tivar 1000® (Poly Hi Solidue, Inc.), hardened polymers, steel and other metals, metal alloys, plastics, nylon, aluminum, fiberglass, aramide fibers, graphite fibers, epoxy resins, polyester resins, polyurethane resins, acrylic resins, carbon-fiber composite, and the like. For example, the prosthetic ankle 12 can be constructed of impregnated glass fibers or carbon fibers in a matrix of polymeric synthetic resin. Moreover, the prosthetic ankle 12 can be constructed of two or more layers of the same types of material or of two or more different types of material. For example, the prosthetic ankle 12 can be comprised of a first layer of material positioned concentrically inside a second layer of material. Also, the first and second layers of material can be spaced-apart from one another or can be in contact with one another.

As shown in FIG. 3, the prosthetic ankle assembly 10 also preferably includes an upper attachment plate 16, an upper insert nut 20, a lower attachment plate 26, a lower insert nut 30, and a link assembly 55. The upper attachment plate 16 is connectable to the lower end 53 of the pylon 52 (e.g., as shown in FIGS. 5A-6C). Preferably, the upper attachment plate 16 includes a shaft 17 or other extension that is shaped for attachment to the lower end 53 of the pylon 52, such as through a male/female mating connection. Alternatively, the lower end 53 of the pylon 52 can include a male extension that is inserted into a female aperture within the prosthetic ankle assembly 10. Preferably, the upper insert nut 20 couples the upper attachment plate 16, and thus, the pylon 52, to the upper leg 14 of the prosthetic ankle 12. Also preferably, the upper insert nut 20 is positioned within a hole 21 in the upper leg 14, within a hole 19 in the upper attachment plate 16, and is secured thereto via a bolt 18 or other fastener. A lock washer 22 is preferably coupled between the upper insert nut 20 and the upper leg portion 14 in order to prevent the upper insert nut 20 from turning when the bolt 18 is tight. Also, the pylon 52 can be integral with the prosthetic ankle assembly 10, as will be described in detail below.

Although the prosthetic ankle assembly 10 is shown and described as using an upper attachment plate 16 and an upper insert nut 20 to couple the prosthetic ankle assembly 10 to the pylon 52, the prosthetic ankle assembly 10 can be attached to the pylon 52 in any suitable manner. Specifically, not all of the embodiments require the upper attachment plate 16, the lock washer 22, the upper insert nut 20, and the bolt 18 to couple the prosthetic ankle assembly 10 to the pylon 52. For example, any number of releasable or non-releasable fasteners can be used to couple the prosthetic ankle assembly 10 to the pylon 52, such as bolts, screws, buckles, clips, mating pins and apertures, nails, rivets, threaded connections, snap-fit connections, press-fit connections, and the like. Similarly, adhesives or resins (e.g., epoxy or silicone), cohesive bonding material, welds, and brazing can be used to couple the prosthetic ankle assembly 10 to the pylon 52. Moreover, various embodiments could employ none, one, or some of these fasteners and methods of attachment in different manners of connection.

The lower attachment plate 26 is preferably coupled to the lower leg 24 of the prosthetic ankle 12 with fasteners, such as screws or bolts 28. The lower attachment plate 26 is preferably connectable to a prosthetic foot 54 (e.g., as shown in FIGS. 5A-6C) via the lower insert nut 30. The lower insert nut 30 is preferably positioned within a hole 31 in the lower leg 24 and a hole 33 in the lower attachment plate 26. The lower insert nut 30 is preferably secured to a heel portion 58 of the prosthetic foot 54 (as shown in FIGS. 5A-6C) by being positioned in a hole in the heel portion 58. The heel portion 58 can also include two or more holes within which the lower insert nut 30 is positionable, so that the position of the prosthetic foot 54 with respect to the prosthetic ankle 12 can be adjusted. Also, the prosthetic ankle assembly 10 and/or the prosthetic ankle 12 can be integral with the prosthetic foot 54 as will be described in detail below.

Although the prosthetic ankle assembly 10 is shown and described as using a lower attachment plate 26, a lower insert nut 30, and screws 28 to couple the prosthetic ankle assembly 10 to the prosthetic foot 54, the prosthetic ankle assembly 10 can be attached to the prosthetic foot 54 in any suitable manner. Specifically, not all of the embodiments require the lower attachment plate 26, the lower insert nut 30, and the screws 28 to couple the prosthetic ankle assembly 10 to the prosthetic foot 54. For example, any number of releasable or non-releasable fasteners can be used to couple the prosthetic ankle assembly 10 to the prosthetic foot 54, such as bolts, screws, buckles, clips, mating pins and apertures, nails, rivets, threaded connections, snap-fit connections, press-fit connections, and the like. Similarly, adhesives or resins (e.g., epoxy or silicone), cohesive bonding material, welds, and brazing can be used to couple the prosthetic ankle assembly 10 to the prosthetic foot 54. Moreover, various embodiments could employ none, one, or some of these fasteners and methods of attachment in different manners of connection. In addition, the manner in which the pylon 52 is coupled to the prosthetic ankle assembly 10 and the manner in which the prosthetic foot 54 is coupled to the prosthetic ankle assembly 10 can be identical in some embodiments or can be different in other embodiments. Finally, the prosthetic foot 54 can be adjustable with respect to the prosthetic ankle 12 in any suitable manner. For example, the lower leg 24 can include an elongated recess and the prosthetic foot 54 can include an elongated rail positionable within the elongated recess, so that the prosthetic foot 54 can slide along the elongated recess with respect to the prosthetic ankle 12. Once the prosthetic foot 54 is positioned, a bolt can be used to secure the prosthetic foot 54 with respect to the prosthetic ankle 12. The ability to adjust the position of the prosthetic foot 54 with respect to the prosthetic ankle 12 allows the amputee to adjust the flexure characteristics of the prosthetic ankle assembly 10 to suit his or her needs.

The interconnecting portion 32 of the prosthetic ankle 12 is preferably integral with the upper leg 14 and the lower leg 24 in order to interconnect the upper leg 14 and the lower leg 24. In other embodiments, other mechanisms such as rigid or resilient linkages, cushions, bars, strips, connectors, and the like, may also be used to provide a connection between the upper leg 14 and the lower leg 24 and to bias the upper leg 14 and the lower leg 24 apart from one another. Although the interconnecting portion 32, the upper leg 14, and the lower leg 24 are preferably arranged to define a C-shaped prosthetic ankle 12, it should be noted that these elements can be arranged in any other manner to define any other ankle shape in which the upper and lower portions are resiliently biased away from one another by an interconnecting portion.

The interconnecting portion 32 is preferably constructed to resiliently bias the upper leg 14 and the lower leg 24 apart from one another. In some embodiments such as that shown in FIGS. 5A-6C, the interconnecting portion 32 is designed to flex about an axis that lies in the medial/lateral plane (also referred to as the frontal or coronal plane) with respect the amputee's body, i.e., a medial/lateral axis 34. However, the interconnecting portion 32 is preferably also constructed to only bias the upper leg 14 and the lower leg 24 apart from one another until the upper leg 14 and the lower leg 24 are substantially parallel (as shown in FIG. 5A). As a result, the upper leg 14 and the lower leg 24 are positioned in a parallel, spaced-apart relationship when most of the amputee's weight W is not placed on the prosthetic ankle 12 (i.e., the low-load, parallel position). Thus, the interconnecting portion 32 biases the upper leg 14 and the lower leg 24 apart from one another, but preferably the upper leg 14 and the lower leg 24 do not flex apart from one another past the low-load, parallel position. In other embodiments, the upper leg 14 and the lower leg 24 can be positioned at an angle (e.g., greater than parallel or less than parallel) relative to one another when most of the amputee's weight W is not placed on the prosthetic ankle 12.

In some embodiments, the interconnecting portion 32 is constructed of a carbon-fiber composite. In order to bias the upper leg 14 and the lower leg 24 apart from one another, the fibers in the interconnecting portion 32 can be orientated at various angles. For example, and with reference to FIG. 3, the angle of the carbon fibers in the interconnecting portion 32 may vary from being parallel with the upper leg 14 and the lower leg 24 at an inner surface 37 of the interconnecting portion 32 to being perpendicular with the upper leg 14 and the lower leg 24 at an outer surface 39 of the interconnecting portion 32. This type of carbon fiber orientation can also be employed for superior strength, resilience, and flexibility with other types of fiber-based materials. In general, the orientation of the material's fibers (or the orientation of other micro-structural elements or the crystalline structure for non-fiber based materials) can vary based upon the location of the fibers or the micro-structural elements in the prosthetic ankle 12 in order to provide a prosthetic ankle 12 having superior strength, resiliency, and flexibility. In other embodiments, other carbon-fiber composite construction techniques, such as imbedding carbon filaments in epoxy resin, resin transfer molding (RTM), and the like, can be used for the construction of the prosthetic ankle 12 and its interconnecting portion 32.

In addition to contributing to the flexure characteristics of the prosthetic walking system 50 at heel-strike (as shown in FIG. 5B), the interconnecting portion 32 can also contribute to the flexure characteristics of the prosthetic walking system 50 at toe-off (i.e., when the amputee's weight W is placed on a toe portion 56 of the prosthetic foot 54, as shown in FIG. 5A). Specifically, due to the biasing of the interconnecting portion 32, the flexure characteristics experienced by the amputee at toe-off are substantially determined by the flexure characteristics of the toe portion 56 of the prosthetic foot 54, as will be described in more detail below.

The prosthetic ankle 12 can be designed according to the desired response to the amputee's weight W compressing the upper leg 14 and the lower leg 24 toward one another, based upon the desired response to the pylon 52 leaning to one side (i.e., canting), and most preferably based upon the desired response to both compression and canting. The prosthetic ankle 12 is preferably designed to include one or more weakened portions that have enhanced flexibility due to the shape or structure of the weakened portions or the materials used to construct the weakened portions. For example, the weakened portions can have reduced widths and/or thinner cross-sections. In addition, the weakened portions can be formed by removing material by perforating the weakened portions with holes, by scoring or grooving the material of the weakened portion, or by notching one or both of the sides of the weakened portion. Also, the weakened portions can be formed by changing the material properties of the weakened portions, such as by changing the orientation of the fibers or by making the material less dense, less stiff, or less hard.

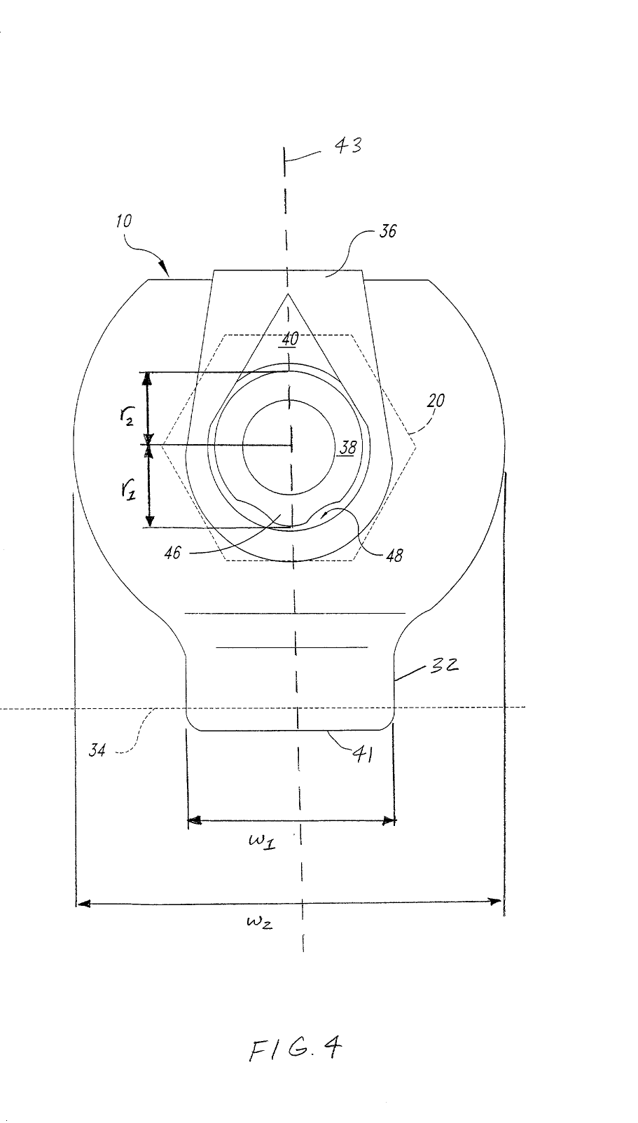

In some embodiments, as shown in FIGS. 3 and 4, the interconnecting portion 32 preferably includes a weakened portion 41 having a width w₁, that is less than a width w₂ of the upper leg 14 and the lower leg 24. The width of the interconnecting portion 32 can gradually vary from the width w₁, of the weakened portion 41 to the width w₂ of the upper leg 14 and the lower leg 24. In other embodiments, the entire interconnecting portion 32 has the width w₁. The weakened portion 41 allows the interconnecting portion 32 to flex about an axis that lies in the anterior/posterior plane (also referred to as the sagittal or median plane) with respect to the amputee's body, i.e., an anterior/posterior axis 43. As a result, when the amputee leans to one side, the interconnecting portion 32 flexes about the anterior/posterior axis 43 allowing the pylon 52 attached to the upper attachment plate 16 to tilt or lean (i.e., cant) with respect to the prosthetic foot 54 attached to the lower attachment plate 26.

The width w₁ of the weakened portion 41 can be adjusted to optimize the canting of the pylon 52 about the anterior/posterior axis 43 according to the amputee's gait or activity type and level. For example, as shown in FIGS. 6B and 6C, a depth d₁ of a medial side 47 of the interconnecting portion 32 can be greater or less than a depth d₂ of a lateral side 49 of the interconnecting portion 32. If the depth d₁ is greater than the depth d₂, the interconnecting portion 32 allows the pylon 52 and the upper leg 14 to cant in the medial direction more easily than in the lateral direction. Conversely, if the depth d₁ is less than the depth d₂, the interconnecting portion 32 allows the pylon 52 and the upper leg 14 to cant in the lateral direction more easily than in the medial direction. Alternatively, the weakened portion 41 can be optimized for the desired canting of the pylon 52 by removing material from either side of the weakened portion 41 (e.g., by perforating the weakened portion 41with holes, by scoring or grooving the material of the weakened portion 41, or by notching one side of the weakened portion 41). Also, the weakened portion 41 can be optimized for the desired canting of the pylon 52 by changing the material properties of either side of the weakened portion 41 (e.g., by changing the orientation of the fibers on one side or by making the material less dense, less stiff, or less hard on one side).

The weakened portion 41 of the interconnecting portion 32 can also be shaped or otherwise constructed to allow the pylon 52 and the upper leg 14 to twist in axial torsion about a longitudinal axis 45 (as designated in FIGS. 5A, 5B, 6B, and 6C) of the pylon 52 with respect to the lower leg 24 and the prosthetic foot 54. For example, the pylon 52 and the upper leg 14 can twist medially with respect to the longitudinal axis 45, while the lower leg 24 and the prosthetic foot 54 remain in a lateral position on the ground. The diameters and the widths of the interconnecting portion 32 can also be adjusted to optimize the ability of the prosthetic ankle 12 to twist in axial torsion about the longitudinal axis 45 according to the amputee's gait or activity level and type. For example, an amputee that golfs or plays baseball may want to optimize the axial torsion capability of the prosthetic ankle 12 so that the prosthetic ankle 12 twists appropriately when he or she swings a golf club or a baseball bat. For example, the weakened portion 41 can be optimized for axial torsion by appropriately reducing the width and/or the cross-section of the weakened portion 41. In addition, the weakened portion 41 can be optimized for axial torsion by appropriately removing material from the weakened portion 41 (e.g., by perforating the weakened portion 41 with holes, by scoring or grooving the material of the weakened portion 41, or by notching the weakened portion 41). Also, the weakened portion 41 can be optimized for axial torsion by appropriately changing the material properties of the weakened portion 41 (e.g., by changing the orientation of the fibers or by making the material less dense, less stiff, or less hard).

In general, the weakened portion 41 can be shaped, can have its material properties selected, or can otherwise be designed to respond as desired to forces in any of the three axes (i.e., bending or flexing, canting, and twisting).

In some embodiments, the weakened portion 41 having the reduced width w₁ of the prosthetic ankle 12 is positioned asymmetrically with respect to the longitudinal axis 45 of the pylon 52. In such embodiments, the weakened portion 41 is preferably positioned asymmetrically according to whether the prosthetic walking system 50 is designed for attachment to the left or right side of the amputee. For example, the weakened portion 41 can be positioned laterally with respect to the longitudinal axis 45 in order to allow the pylon 52 to cant more easily in the medial direction. Alternatively, the weakened portion 41 can be positioned medially with respect to the longitudinal axis 45 in order to allow the pylon 52 to cant more easily in the lateral direction. In this regard, the interconnecting portion 32 need not be axially aligned with the upper leg 14 and/or the lower leg 24 as illustrated in FIGS. 3-7B.

Some embodiments of the present invention employ a link assembly 55 to at least partially define a maximum displacement between the upper leg 14 and the lower leg 24. In some embodiments, as shown in FIGS. 3-6C, the link assembly 55 is comprised of a strap 36 coupled between the upper leg 14 and the lower leg 24. In opposition to the biasing of the interconnecting portion 32, the strap 36 preferably limits the upper leg 14 and the lower leg 24 from flexing apart from one another about the medial/lateral axis 34 beyond the low-load, parallel position (as shown in FIG. 5A) or beyond any other desired relative angular position of the upper leg 14 and the lower leg 24. The strap 36 preferably includes an upper aperture 40 and a lower aperture 44. The upper aperture 40 and the lower aperture 44 can have any suitable shape, such as a tear-drop shape as shown in FIG. 3. Preferably, a shaft 38 of the upper insert nut 20 extends through the upper aperture 40, and a shaft 42 of the lower insert nut 30 extends through the lower aperture 44.

Any other manner of connecting the strap 36 to limit the displacement between the upper leg 14 and the lower leg 24 can be employed. The strap 36 can be attached to or coupled between any suitable combination of the upper leg 14, the lower leg 24, the pylon 52, the prosthetic foot 54, the upper attachment plate 16, and the lower attachment plate 26. For example, the strap 36 can be coupled between the upper attachment plate 16 and the lower attachment plate 26. Also for example, the strap 36 can be clamped to the upper leg 14 and the lower leg 24 using any type of clamp device. The strap 36 can also be attached to the suitable components using adhesives, cohesive bonding materials, welds, brazing, and the like. Any other manner of connection of the strap 36 that results in limiting the displacement between the upper leg 14 and the lower leg 24 falls within the spirit and scope of the invention.

In some embodiments, the strap 36 is constructed of a rigid, non-resilient, flexile material, such as Kevlar® (E.I. du Pont de Nemours and Company). In other embodiments, other resilient flexile materials, such as nylon or a phenolic-fiber material, can be used for the construction of the strap 36. In still other embodiments, the strap 36 can be constructed of a less rigid, flexible material that generates an increasing resistance between the upper leg 14 and the lower leg 24 as the upper leg 14 and the lower leg 24 spread farther apart from one another between heel-strike and toe-off.

In some embodiments, the strap 36 can be selected from a number of available straps, each one having different lengths and/or different material properties. Each one of the available straps can be designed for different amputee gait preferences, different amputee activity types or levels, different amputee weight levels, and the like.

With reference to FIG. 3, a resilient bias element, such as a cushion 35, can be positioned between the upper leg 14 and the lower leg 24. The cushion 35 assists the interconnecting portion 32 in resiliently biasing the upper leg 14 and the lower leg 24 apart from one another. In other embodiments, other resilient bias elements, such as air bladders, liquid bladders, and springs, can be used for assisting in biasing the upper leg 14 and the lower leg 24 apart from one another. As best shown in FIG. 5A, the cushion 35 is preferably positioned between the posterior ends of the upper leg 14 and the lower leg 24 adjacent to the strap 36, although the cushion 35 can be positioned in any location between the upper and lower legs 24 to perform this same function. Different cushions 35 can be used for different system performance. For example, different cushions 35 can have different dimensions (e.g., thickness, width, or length), different stiffnesses, constant and non-constant spring coefficients, and the like. The cushion 35 can also be replaced for different amputee gait preferences, different amputee activity types or levels, different amputee weight levels, and the like. The dimensions and properties of the cushion 35 can vary across any dimension to generate similar results to those discussed above with reference to the weakened portion 41. The cushion 35 can also be moved and secured in different locations between the upper leg 14 and the lower leg 24 to generate different resistances and reactions to compression, canting, and twisting of the prosthetic ankle 12 based upon different amputee gait preferences, different amputee activity types or levels, different amputee weight levels, and the like.

With reference to the embodiment illustrated in FIG. 4, the shaft 38 of the upper insert nut 20 preferably includes a cam lobe 46 having a radius r₁ that is greater than a radius r₂ of the remainder of the shaft 38. The strap 36 includes an interior edge 48. The cam lobe 46 of the upper insert nut 20 engages the interior edge 48 of the strap 36 in order to vary the tension of the strap 36. The engagement between the cam lobe 46 and the interior edge 48 of the strap 36 (as defined by the adjustable rotational position of the shaft 38) determines the tension of the strap 36 and assists in defining the maximum displacement between the upper leg 14 and the lower leg 24.

In use, as shown in FIGS. 5A-6C, the prosthetic ankle assembly 10 is coupled between the pylon 52 and the prosthetic foot 54 in the prosthetic walking system 50. As shown in FIG. 5A, when most of the amputee's weight W is placed on the toe portion 56 of the prosthetic foot 54 and not on the prosthetic ankle 12, the interconnecting portion 32 of the prosthetic ankle 12 biases the upper leg 14 and the lower leg 24 apart from one another to preferred relative positions, such as to a substantially parallel relationship as illustrated. However, the strap 36 prevents the upper leg 14 and the lower leg 24 from being biased apart from one another past the low-load (e.g., parallel) position as shown in FIG. 5A.

The maximum displacement between the upper leg 14 and the lower leg 24 is determined in large part by the materials used in the construction of the interconnecting portion 32 and the strap 36. For example, the maximum displacement can be achieved by constructing the interconnecting portion 32 of a material that provides greater biasing and also constructing the strap 36 of a material that provides greater tension. Alternatively, the maximum displacement can be achieved by constructing the interconnecting portion 32 of a material that provides less biasing and also constructing the strap 36 of a material that provides less tension.

In addition, the tension of the strap 36 (due to the position of the cam lobe 46 in engagement with the internal surface 48 of the upper insert nut 20 in the illustrated preferred embodiment of FIGS. 3-7B) also assists in defining the maximum displacement between the upper leg 14 and the lower leg 24. For example, if the position of the cam lobe 46 places the strap 36 in high tension and the prosthetic ankle 12 is substantially rigid, the flexure characteristics of the prosthetic walking system 50 at toe-off (as shown in FIG. 5A) are determined in increasing part by the flexure characteristics of the toe portion 56 of the prosthetic foot 54. Conversely, if the position of the cam lobe 46 places the strap 36 in low tension and the prosthetic ankle 12 is more flexible, the flexure characteristics of the prosthetic walking system 50 at toe-off are determined to a greater degree by the flexure characteristics of the prosthetic ankle 12.

As shown in FIG. 5B, as the heel portion 58 of the prosthetic foot 54 contacts the ground (i.e., at heel-strike) and most of the amputee's weight W is placed on the prosthetic ankle 12, the interconnecting portion 32 flexes about the medial/lateral axis 34 and a posterior end 51 of the upper leg 14 flexes downward toward a posterior end 57 of the lower leg 24. When the interconnecting portion 32 flexes on heel-strike, a bottom surface 65 of the prosthetic foot 54 is preferably quickly forced downward into contact with the ground. Thus, the amputee's weight W is supported by the bottom surface 65 of the prosthetic foot 54 for most of the amputee's gait. As a result, the amputee experiences better stability while using the prosthetic walking system 50.

The prosthetic walking system 50 preferably also accommodates an amputee's movements in the medial and/or lateral directions. For example, FIGS. 6A-6C illustrate operation of the prosthetic walking system 50 when the prosthetic walking system 50 is positioned on the amputee's right side and the amputee moves in the medial direction (to the right as viewed in FIG. 6A, wherein the amputee is facing the viewer). More specifically, FIGS. 6A-6C illustrate the operation of the prosthetic walking system 50 when the prosthetic foot 54 is flat on the ground just after heel-strike and the amputee leans in the medial direction. Although FIGS. 6A-6C only illustrate the operation of the prosthetic walking system 50 when the prosthetic walking system 50 is positioned on the amputee's right side and when the amputee moves in the medial direction, the prosthetic walking system 50 can, of course, be positioned on the amputee's left side.

FIG. 6A is a front elevational view of the prosthetic walking system 50 positioned on the amputee's right side with the amputee facing the viewer. As shown in FIG. 6A, the prosthetic foot 54 is flat on the ground, and the amputee is leaning to his or her left. The interconnecting portion 32 is flexed toward the medial direction, so that the upper leg 14 of the prosthetic ankle 12 is rotated about the anterior/posterior axis 43 (as designated in FIG. 4) in the medial direction. Accordingly, the distance between a medial side 75 of the upper leg 14 and a medial side 76 of the lower leg 24 is less than the distance between a lateral side 77 of the upper leg 14 and a lateral side 78 of the lower leg 24. As the interconnecting portion 32 flexes and the upper leg 14 rotates in this manner, the pylon 52 is allowed to cant in the medial direction with respect to the prosthetic foot 54.

FIGS. 6A-6C could instead illustrate the prosthetic walking system 50 according to the present invention for a left leg of an amputee (with the amputee facing the viewer in FIG. 6A), in which case the amputee is leaning or moving laterally to the right in FIG. 6A, and in which case the upper leg 14 is flexed to rotate about the anterior/posterior axis 43 in an opposite direction to that described immediately above.

FIG. 6B is a medial side elevational view of the prosthetic walking system 50 of FIG. 6A (wherein the prosthetic walking system 50 is on the right side of an amputee facing the viewer as described above). As shown in FIG. 6B, the upper leg 14 lies along an anterior/posterior axis 60, and the upper leg 14 includes a medial posterior portion 64 and a medial anterior portion 67. Similarly, the lower leg 24 lies along an anterior/posterior axis 62, and the lower leg 24 includes a medial posterior portion 66 and a medial anterior portion 68. When the pylon 52 cants in the medial direction, the upper leg 14 preferably twists about the axis 60 and the lower leg 24 twists about the axis 62. In other embodiments, only the upper leg 14 twists about the axis 60 or only the lower leg 24 twists about the axis 62. The medial posterior portion 64 of the upper leg 14 and the medial posterior portion 66 of the lower leg 24 are each positioned further away from the interconnecting portion 32 than the medial anterior portion 67 of the upper leg 14 and the medial anterior portion 68 of the lower leg 24. Accordingly, the medial posterior portion 64 of the upper leg 14 preferably twists downwardly more than the medial anterior portion 67, and the medial posterior portion 66 of the lower leg 24 preferably twists upwardly more than the medial anterior portion 68. In other embodiments (and depending upon the shape and material properties of the prosthetic ankle 12 as described above), the medial posterior portion 64 of the upper leg 14 can twist upwardly more than the medial anterior portion 67, and the medial posterior portion 66 of the lower leg 24 can twist downwardly more than the medial anterior portion 68. In still other embodiments, the medial posterior portion 64 of the upper leg 14 can twist upwardly the same distance as the medial anterior portion 67, and the medial posterior portion 66 of the lower leg 24 can twist downwardly the same distance as the medial anterior portion 68. In these embodiments, the canting of the pylon 52 would be enabled or primarily enabled by the twisting of the interconnecting portion 32, rather than the upper leg 14 and the lower leg 24.

FIG. 6C is a lateral side elevational view of the prosthetic walking system 50 of FIGS. 6A and 6B (wherein the prosthetic walking system 50 is on the right side of an amputee facing the viewer as described above). As shown in FIG. 6C, as a result of the twisting of the upper leg 14 about the axis 60 and the lower leg 24 about the axis 62, a lateral anterior portion 69 on the lateral side 78 of the lower leg 24 is forced downwardly with a force Fₐ. Moreover, the downward force Fₐ on the lateral anterior portion 69 of the lower leg 24 causes a corresponding downward force F_{b} on a lateral side 79 of the toe portion 56 of the prosthetic foot 54. Due to the forces Fₐ and F_{b}, the prosthetic foot 54 “digs in” toward the ground, and thus is more stable when the amputee leans in the medial direction.

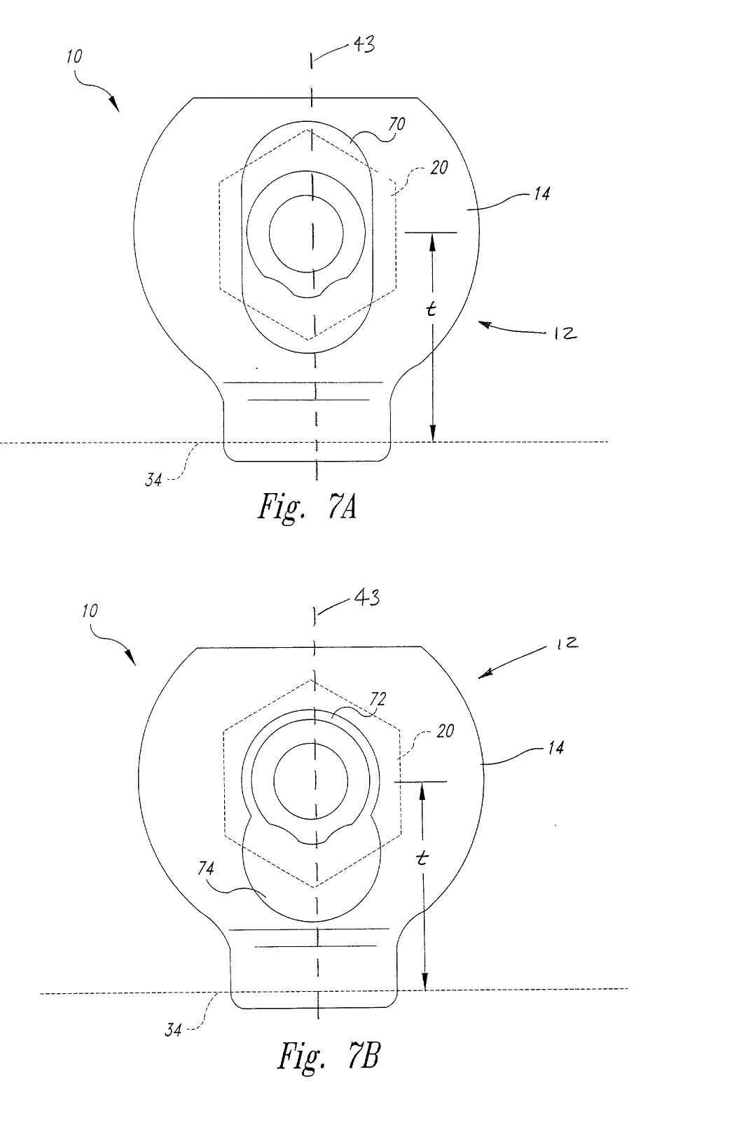

FIG. 7A illustrates an alternative embodiment of the prosthetic ankle 12. As shown in FIG. 7A, the upper insert nut 20 is inserted into a slotted aperture 70 in the upper leg 14. The pylon 52 (as shown in FIGS. 5A-6C) can be positioned at various positions within the slotted aperture 70 in order to vary a torque-arm distance t between the medial/lateral axis 34 and the longitudinal axis 45 (as designated in FIGS. 5A-6C) of the pylon 52. The ability to adjust the torque arm distance t allows the amputee to adjust the flexure characteristics of the prosthetic ankle assembly 10 to suit his or her needs.

FIG. 7B illustrates another alternative embodiment of the prosthetic ankle 12. The upper leg 14 includes an aperture comprised of a first lobe 72 and a second lobe 74. Although the first lobe 72 and the second lobe 74 are shown in FIG. 7B as being partially joined, the first lobe 72 and the second lobe 74 may also be separated by a portion of the upper leg 14. The first lobe 72 corresponds to a first position for the pylon 52, and the second lobe 74 corresponds to a second position for the pylon 52. As shown in FIG. 7B, the upper insert nut 20 is positioned within the first lobe 72. By selecting either the first lobe 72 or the second lobe 74, the amputee can adjust the torque arm distance t between the medial/lateral axis 34 and the longitudinal axis 45 of the pylon 52. Thus, the amputee can adjust the flexure characteristics of the prosthetic ankle assembly 10 to suit his or her needs.

The manner in which the pylon 52 is adjustable with respect to the prosthetic ankle assembly 10 can depend on the manner in which the pylon 52 is coupled to the prosthetic ankle assembly 10. For example, if the pylon 52 includes a male extension that is positionable within a female aperture in the prosthetic ankle assembly 10, different apertures or connections at different longitudinal and lateral positions can be formed in the upper leg 14 of the prosthetic ankle 12. Moreover, the pylon 52 can be releasably or permanently coupled, attached, or otherwise connected in different longitudinal and lateral positions to the upper leg 14. For example, the pylon 52 can be attached using welding, brazing, setscrewing, pinning, locking, clipping, and the like to secure the lower end 53 of the pylon 52 to a desired position in a longitudinal or lateral track on or connected to the upper leg 14.

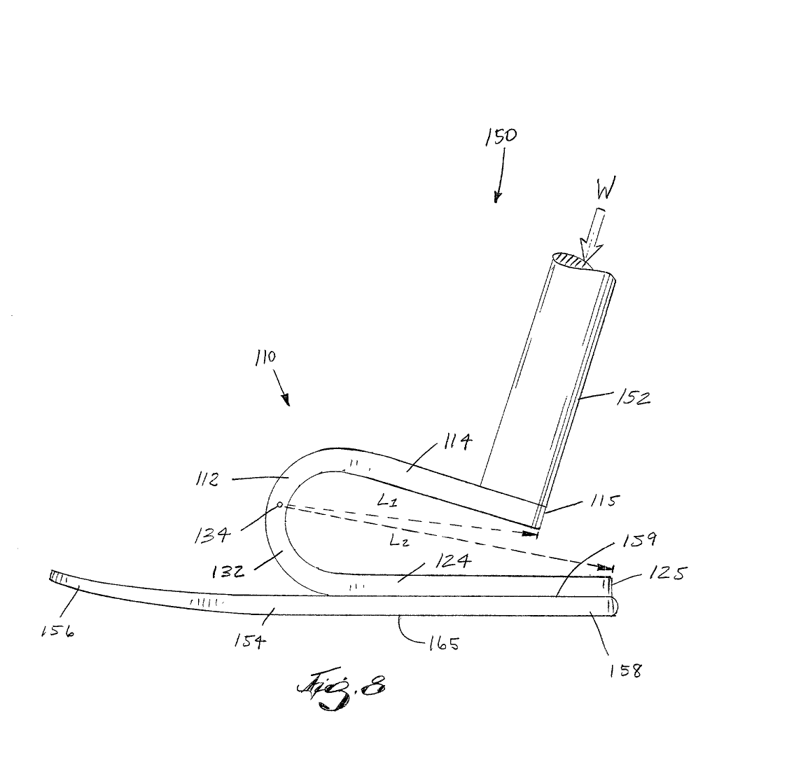

FIG. 8 illustrates a prosthetic walking system 150 which is an alternative embodiment of the prosthetic walking system 50 shown in FIGS. 3-6C. Elements and features of the prosthetic walking system 150 illustrated in FIG. 8 having a form, structure, or function similar to that found in the prosthetic walking system of FIGS. 3-6C are given corresponding reference numbers in the 100 series. The prosthetic walking system 150 includes a prosthetic ankle assembly 110 which is comprised of a prosthetic ankle 112 coupled between a pylon 152 and a prosthetic foot 154. The prosthetic ankle 112 preferably includes an upper leg 114, an interconnecting portion 132, and a lower leg 124. The lower leg 124 is preferably joined to an upper surface 159 of a heel portion 158 of prosthetic foot 154 with adhesive or cohesive bonding material (e.g., epoxy adhesives or resins or silicone adhesives), rivets, bolts and nuts, screws, other threaded fasteners and the like. Any manner described above for connecting the pylon to the upper leg of the embodiment illustrated in FIGS. 3-6C can be used here to connect the upper leg 114 to the pylon 152 and to connect the lower leg 124 to the heel portion 158 of the prosthetic foot 154. Preferably, the interconnecting portion 132 flexes about a medial/lateral axis 134.

The upper leg 114 has a length L₁ defined between the medial/lateral axis 134 and a posterior end 115 of the upper leg 114. The lower leg 124 has a length L₂ defined between the medial/lateral axis 134 and a posterior end 125 of the lower leg 124. Preferably, the length L₂ is greater than the length L₁, so that the lower leg 124 is longer than the upper leg 114. As a result, the lower leg 124 has a greater torque-arm moment (i.e., a greater tendency to produce motion about the medial/lateral axis 134) than the upper leg 114. When the amputee's weight W is pressed down on the prosthetic walking system 150 at heel-strike, the greater torque-arm moment of the lower leg 124 causes the lower leg 124 to flex upwardly toward the upper leg 114 to a greater extent than the upper leg 114 flexes downwardly toward the lower leg 124. The flexion of the lower leg 124 upwardly about the medial/lateral axis 134 causes a toe portion 156 of prosthetic foot 154 to be driven downwardly. As a result, a bottom surface 165 of the prosthetic foot 154 is in contact with the ground immediately after heel-strike and preferably during most of the amputee's gait. The principles and features of the embodiment shown and described with respect to FIG. 8 can be employed with any of the other embodiments of the present invention described herein.

Individual components for a pylon, a prosthetic ankle, and a prosthetic foot can each be constructed separately and then assembled to form a prosthetic walking system (e.g., as in the prosthetic walking system 50 illustrated in FIGS. 5A-6C and the prosthetic walking system 150 illustrated in FIG. 8). However, one of ordinary skill in the art will recognize that two or more of these components can be integrally connected to form a continuous, integral unit. As used herein, the term “integrally connected” means that two or more components formed together into a continuous, integral unit, such as by being extruded, molded, bent, pressed, stamped, cast, sintered, or otherwise formed from a single piece, element, or structure. It will be understood by one of ordinary skill in the art that components can be integrally connected even if the components are constructed of different materials and combined in a manufacturing process, such as through lamination or insert molding or casting, to form the continuous, integral unit. In many cases, a prosthetic walking system comprised of an integral unit is more easily manufactured and is more stable than a prosthetic walking system having multiple separate components. By way of example only, a pylon and a prosthetic ankle can be formed into an integral unit and coupled to a separate prosthetic foot. Also, a prosthetic ankle and a prosthetic foot can be formed into an integral unit and coupled to a separate pylon. In addition, a pylon, a prosthetic ankle, and a prosthetic foot can be formed into an integral unit.

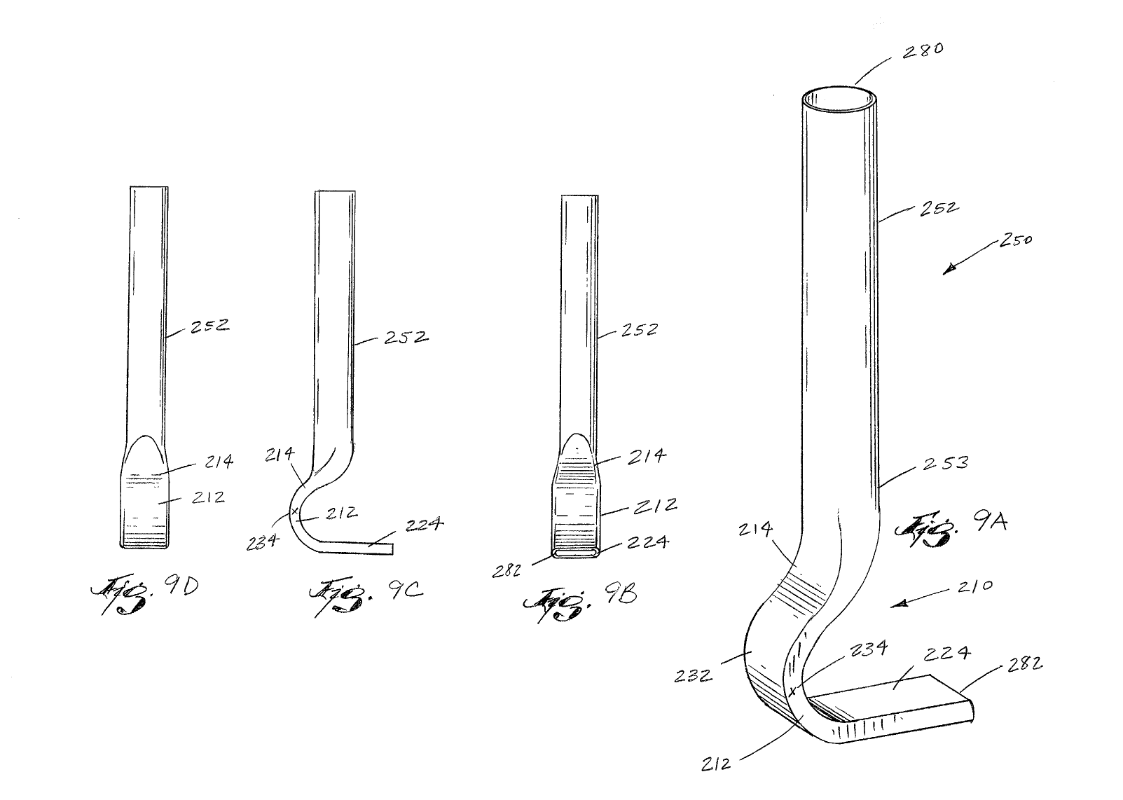

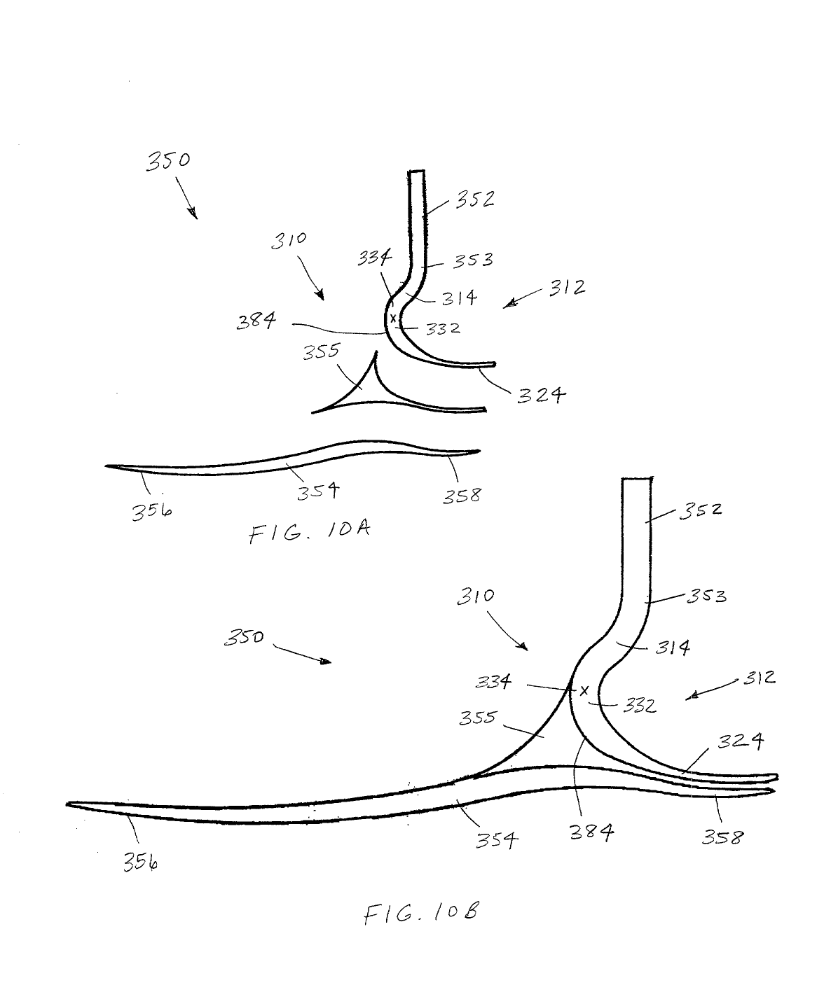

FIGS. 9A-9D illustrate one such continuous, integral unit embodied by a prosthetic walking system 250, which is an alternative embodiment of the prosthetic walking systems 50 and 150. Elements and features of the prosthetic walking system 250 illustrated in FIGS. 9A-9D having a form, structure, or function similar to that found in the prosthetic walking system of FIGS. 3-8 are given corresponding reference numbers in the 200 series. The prosthetic walking system 250 includes a pylon 252 integrally connected to a prosthetic ankle assembly 210. The prosthetic ankle assembly 210 illustrated in FIGS. 9A-9D is comprised of a prosthetic ankle 212. However, the prosthetic ankle assembly 210 can include other components, such as a strap or other elements described above, a link assembly as will be described in detail below, and the like. Each of the links and link assemblies described below are adaptable to fit the prosthetic walking system 250.

With continued reference to FIGS. 9A-9D, a lower end 253 of the pylon 252 is preferably integrally connected to an upper leg 214 of the prosthetic ankle 212. More specifically, the upper leg 214 is preferably integrally connected to an interconnecting portion 232 which is integrally connected to a lower leg 224. By forming an integral unit, the functional characteristics of each of the independent components, including the pylon 252, the upper leg 214, the interconnecting region 232, and the lower leg 224, are combined into a unitary system. The integral unit preferably flexes within the interconnecting portion 232 at a medial/lateral axis 234. Although not shown in FIGS. 9A-9D, the lower leg 224 is preferably coupled to a prosthetic foot.

Preferably, the pylon 252 of the prosthetic walking system 250 has a substantially circular cross-section 280, as best shown in FIG. 9A. Also, the prosthetic ankle 212 preferably has a substantially rectangular cross-section 282, as best shown in FIGS. 9A and 9B. The pylon 252 and the prosthetic ankle 212 can also have other cross-section configurations, such as oval, triangular, rectangular, pentagonal, octagonal, or irregular-shaped cross-sections. The pylon 252 and the prosthetic ankle 212 can each have the same cross-section configuration or can each have a different cross-section configuration. In addition, any portion or all of the pylon 252 and/or the prosthetic ankle 212 can be solid or hollow. Preferably, substantially the entire pylon 252 is hollow, while substantially the entire prosthetic ankle 212 is solid. In the embodiment shown in FIGS. 9A-9D, for example, the pylon 252 is solid as it transitions to the upper leg 214 of the prosthetic ankle 212. Preferably, the lower end 253 of the pylon 252 makes a continuous transition in shape from the circular cross-section 280 to the rectangular cross-section 282 of the prosthetic ankle 212, although other transitions between these sections are possible. The circular cross-section 280 provides the pylon 252 with greater stiffness and a greater cross-sectional area of inertia than the rectangular cross-section 282 of the prosthetic ankle 212. As a result, the prosthetic walking system 250 preferably flexes at the interconnecting portion 232 of the prosthetic ankle 212, rather than at the pylon 252, when the amputee's weight is placed on the prosthetic walking system 250. The cross-section configurations of the pylon 252 and the prosthetic ankle 212 can also be selected in order to optimize the flexion of the interconnecting portion 232.

Due to its integral construction, some embodiments of the prosthetic walking system 250 can be constructed with less material and can be lighter in weight than prosthetic walking systems having separate components. If the prosthetic walking system 250 is constructed of a single type of material, the material must be rigid enough for the pylon 252 to support the amputee's weight and for the interconnecting portion 232 to bias the upper leg 214 and the lower leg 224 apart from one another. However, the material is preferably also resilient enough to allow the upper leg 214 and the lower leg 224 to flex toward one another when the amputee's weight is loading the prosthetic ankle 212.

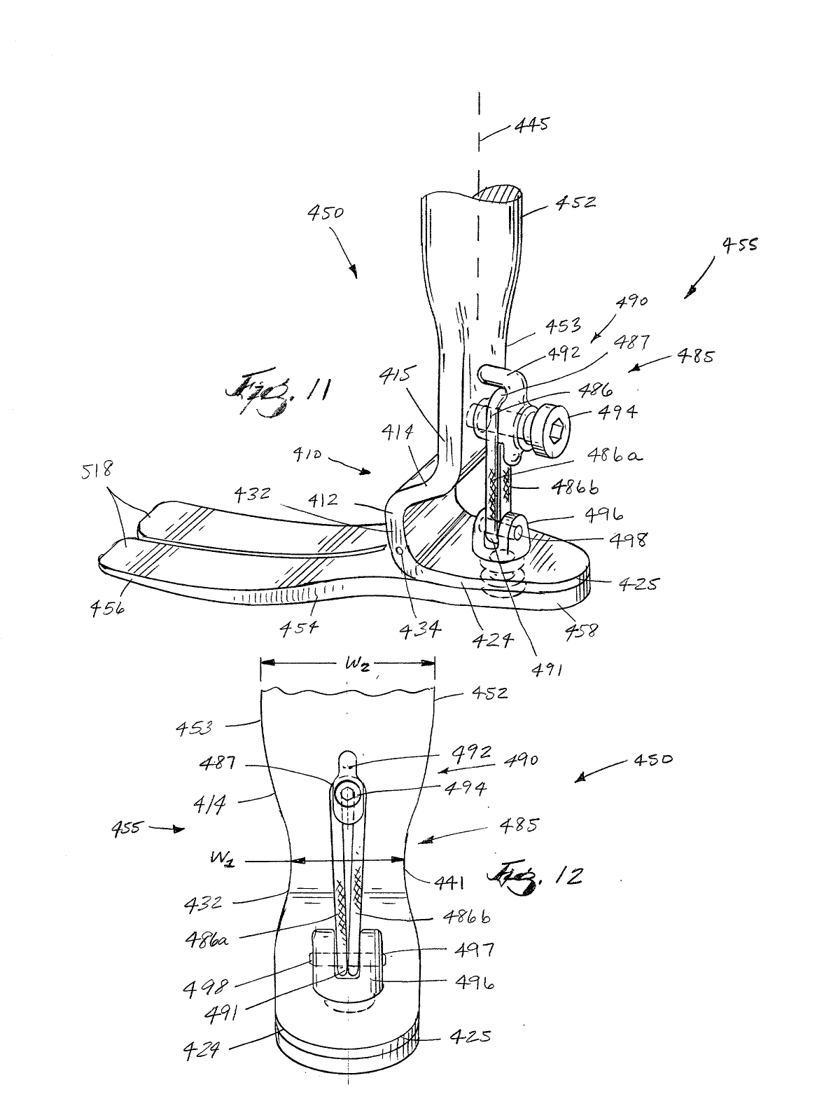

FIGS. 10A and 10B illustrate a prosthetic walking system 350 which is an alternative embodiment of the prosthetic walking systems 50, 150, and 250 described above. Elements and features of the prosthetic walking system 350 illustrated in FIGS. 10A and 10B having a form, structure, or function similar to that found in the prosthetic walking system of FIGS. 3-9D are given corresponding reference numbers in the 300 series. The prosthetic walking system 350 preferably includes a pylon 352 integrally connected to a prosthetic ankle assembly 310. The prosthetic ankle assembly 310 as illustrated in FIGS. 10A and 10B is comprised of a prosthetic ankle 312 and a link 355. A lower end 353 of the pylon 352 is preferably integrally connected to an upper leg 314 of the prosthetic ankle 312. The upper leg 314 is preferably integrally connected to an interconnecting portion 332 which is integrally connected to a lower leg 324. As with the embodiments of the present invention described above, the interconnecting portion 332 preferably flexes at a medial/lateral axis 334.

The link 355 is preferably constructed of a high-durometer elastomer, such as urethane, that is cast into the particular shape shown in FIGS. 10A and 10B. The link 355 is coupled, preferably using high-strength adhesives, to an anterior side 384 of the interconnecting portion 332 of the prosthetic ankle 312. The prosthetic ankle 312 and the link assembly 355 are integrally connected to the prosthetic foot 354, also preferably using high-strength adhesives.

Preferably, the link 355 limits the displacement of the upper leg 314 toward a toe portion 356 of the prosthetic foot 354 and away from the lower leg 324. In other words, the link 355 preferably resists and/or limits the amount of anterior movement of the pylon 352 and the upper leg 314 toward the toe portion 356 of the prosthetic foot 354 as force is increasingly applied to the toe portion 356 during the amputee's gait. Although the shape of the link 355 shown in FIGS. 10A and 10B resists this forward motion by conforming to the prosthetic ankle 312 and to the prosthetic foot 354, the link 355 could have other shapes that also perform this function. For example, the link 355 can be wedge-shaped, round, oval, polygonal, irregularly-shaped, or any other shape suitable for location at an anterior interface between the prosthetic ankle 312 and the prosthetic foot 354. In some embodiments, the link 355 does not fully extend beneath the prosthetic ankle 312 as shown in FIGS. 10A and 10B. The link 355 can be releasably or permanently coupled, connected, or attached to the prosthetic ankle 312 and the prosthetic foot 354 in any of the manners of connection described above with respect to FIGS. 3-6C for connecting the prosthetic ankle and the prosthetic foot.

The prosthetic foot 354 of the prosthetic walking system 350 can be rotated with respect to the medial/lateral axis 334 and coupled to the prosthetic ankle 312 in various positions. For example, the toe portion 356 of the prosthetic foot 354 can be angled downwardly from the position shown in FIGS. 10A and 10B, resulting in a heel portion 358 of the prosthetic foot 354 being raised (i.e., an increase in heel-rise). Alternatively, the toe portion 356 can be angled upwardly from the position shown in FIGS. 10A and 10B, resulting in the heel portion 358 being lowered (i.e., a decrease in heel-rise).

The link 355 employed in the embodiment shown in FIGS. 10A and 10B and the manner in which the link 355 is connected and operates as described above can be employed in any of the other embodiments described herein.

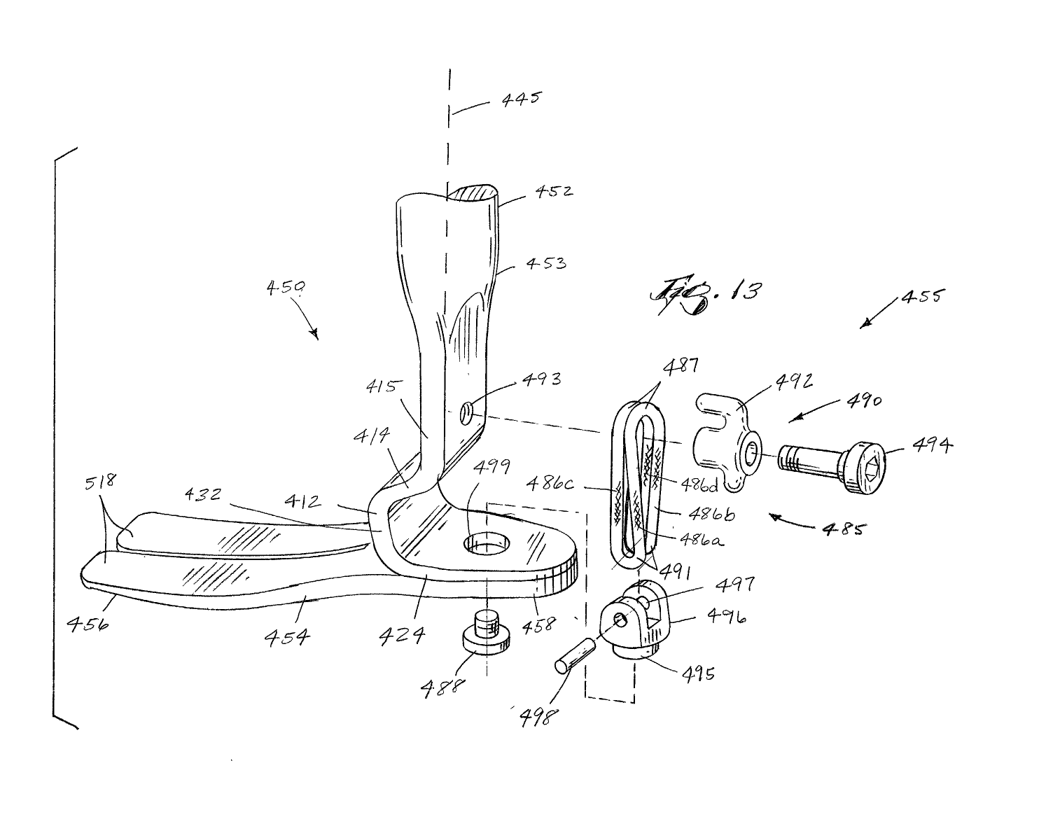

FIGS. 11-13 illustrate a prosthetic walking system 450 which is an alternative embodiment of the prosthetic walking systems 50, 150, 250, and 350 described above. Elements and features of the prosthetic walking system 450 illustrated in FIGS. 11-13 having a form, structure, or function similar to that found in the prosthetic walking system of FIGS. 3-10B are given corresponding reference numbers in the 400 series. The prosthetic walking system 450 includes a pylon 452 integrally connected to a prosthetic ankle assembly 410. In the illustrated preferred embodiment of FIGS. 11-13, the pylon 453, prosthetic ankle 412, and their manner of connection are similar to that of the preferred embodiment illustrated in FIGS. 9A-9D. The prosthetic ankle assembly 410 as illustrated in FIGS. 11 -13 is comprised of a prosthetic ankle 412 and a link assembly 455, as will be described below. A lower end 453 of the pylon 452 is preferably integrally connected to an upper leg 414 of the prosthetic ankle 412. The upper leg 414 is preferably integrally connected to an interconnecting portion 432 which is integrally connected to a lower leg 424. Preferably, the interconnecting portion 432 flexes at a medial/lateral axis 434 as described with respect to the embodiments described above.

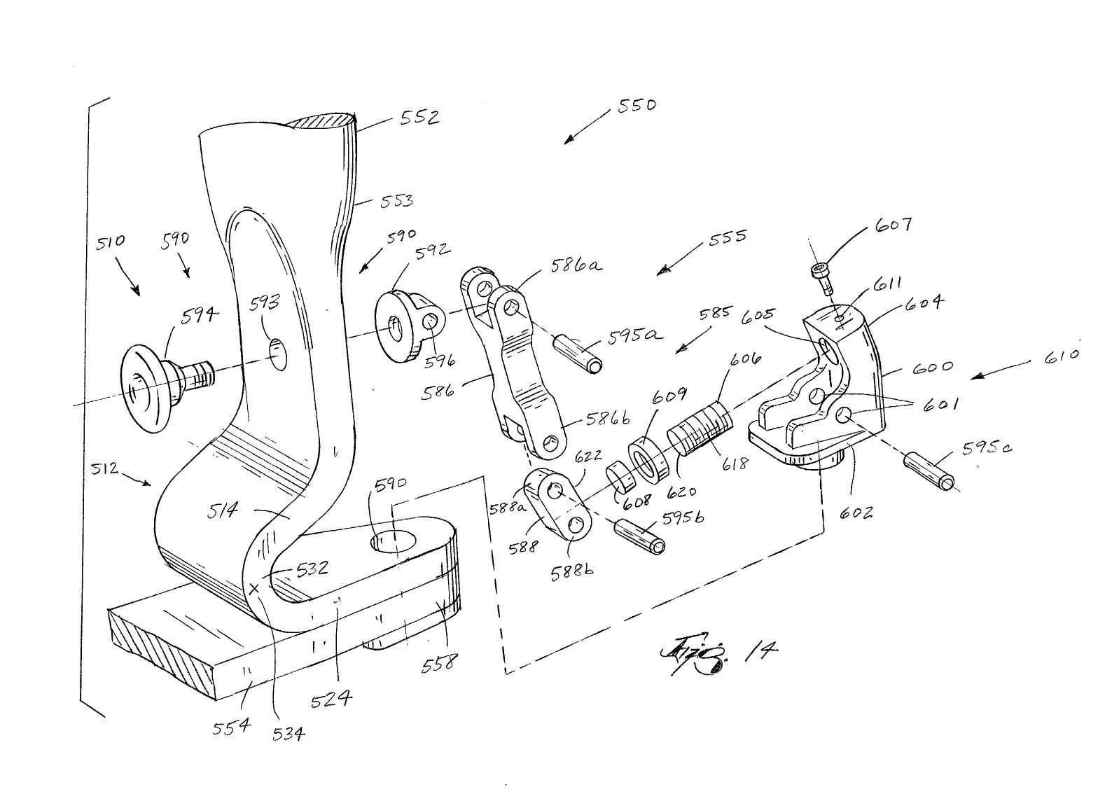

As shown in FIG. 12, the interconnecting portion 432 preferably includes a weakened portion 441 having a width w₁ that is less than a width w₂ of the pylon 452. In addition, the other manners in which the weakened portion can be configured as described and shown with respect the embodiment shown in FIGS. 3-6C can also be used for the weakened portion 441. Preferably, the width of the interconnecting portion 432 gradually varies from the width w₁ of the weakened portion 441 to the width w₂ of the pylon 452, although these dimensions can vary in any other manner desired. The lower leg 424 can have any width, including a width equal to the width w₁ of the weakened portion 441 or the width w₂ of the pylon 452. The weakened portion 441 may also be positioned asymmetrically with respect to a longitudinal axis 445 of the pylon 452. As discussed above with respect to the embodiment shown in FIGS. 3-6C, the weakened portion 441 can be shaped, can have its material properties selected, or can otherwise be designed to respond as desired to forces in any of the three axes (i.e., bending or flexing, canting, and twisting).

FIGS. 11-13 illustrate a limit strap assembly 485, which is one embodiment of a link assembly similar in function to the link assembly 55 shown and described with respect to FIGS. 3-6C. Although only shown and described with respect to the prosthetic walking system 450, the limit strap assembly 485 is also suitable for use with the prosthetic walking systems 50, 150, 250, and 350 described above, and particularly with the prosthetic walking systems 250 and 350 which have an integral construction. The limit strap assembly 485 preferably includes a link in the form of a resilient cord 486 that limits flexion of the upper leg 414 and the lower leg 424 away from one another. The cord 486 can be constructed of one strand of material (e.g., a single strap). The cord 486 can also be constructed of two or more strands, fibers, or filaments of material woven or twisted together and covered with a suitable coating. Moreover, the cord 486 can be comprised of two or more individual lengths of material that are not woven together or covered with a suitable coating. In general, the cord 486 can be constructed of any combination of strands, fibers, filaments, or lengths of material and any combination of these elements can be woven together and/or covered with a suitable coating. The cord 486 can be constructed of any resilient, flexible, abrasion-resistant material that is strong enough to limit the biasing force of the interconnecting region 432. Also, the cord 486 can be constructed of a combination of materials in order to achieve the desired characteristics. For example, materials such as nylon, rubber, polypropylene, polyester, cotton, Nomex®, or Kevlar® (both manufactured by E. I. du Pont de Nemours and Company) can be used alone or in combination to construct the cord 486. Also, the cord 486 can have any degree of flexibility-from not elongating as the amputee's weight shifts between heel-strike and toe-off to easily elongating as the amputee's weight shifts between heel-strike and toe-off. For example, in order to achieve similar results, the cord 486 could be shorter and elongate more easily or the cord 486 could be longer and elongate less easily as the amputee's weight shifts between heel-strike and toe-off.

The resilient cord 486 is preferably connected to define two or more lengths over which the biasing force between the upper leg 414 and the lower leg 424 is distributed. For example, FIGS. 11 and 12 illustrate a cord 486 having two lengths 486a and 486b of resilient material. Similarly, FIG. 13 illustrates a cord 486 having four lengths 486a, 486b, 486c, and 486d of resilient material.

In the illustrated embodiment of FIGS. 11-13, the cord 486 has an upper portion 487 that is wrapped around an upper post assembly 490 and a lower portion 491 that is wrapped around a lower post assembly 496. The upper post assembly 490 is preferably attached at a posterior end 415 of the upper leg 414 or at the lower end 453 of the pylon 452. As best shown in FIG. 13 for example, the upper post assembly 490 is preferably attached to the pylon 452 by an upper bolt 494 being passed through an upper post cover 492 and threaded into a hole 493 in the lower end 453 of the pylon 452. The upper post cover 492 covers the upper bolt 494 in order to protect the cord 486 from the bolt threads. Preferably, the upper post cover 492 is shaped to also cover the upper portion 487 of the cord 486 in order to retain the upper portion 487 of the cord 486 within the upper post assembly 490.

The lower post assembly 496 preferably includes receiving holes 497 that receive a lower post 498. The lower portion 491 of the cord 486 is preferably wrapped around or otherwise secured to the lower post 498. The lower post 498 can be a pin, bolt, or other shaft member that is securely inserted into the receiving holes 497 of the lower post assembly 496. Preferably, the lower post assembly 496 is attached to the lower leg 424 of the prosthetic ankle 412 and/or to the heel portion 458 of the prosthetic foot 454. As best shown in FIG. 13 for example, the lower post assembly 496 is preferably attached to the heel portion 458 of the prosthetic foot 454 via a shaft 495 positioned in a hole 499 through both the lower leg 424 of the prosthetic ankle 412 and the heel portion 458 of the prosthetic foot 454. Moreover, the lower post assembly 496 is preferably further secured to the prosthetic foot 454 via a lower bolt 488 threaded through the heel portion 458 of the prosthetic foot 454 and into a hole (not shown) in the bottom of the shaft 495.

The cord 486 can be attached to the pylon 452 and/or the upper leg 414 and to the lower leg 424 and/or the prosthetic foot 454 in a number of other manners. Specifically, the upper post assembly 490 and the lower post assembly 496 are not necessary in other embodiments. For example, the cord 486 can be attached by being trained about an upper pin or hook and a lower pin or hook. Also, the cord 486 can be attached by being looped about a pin or hook extending from the pylon 452 or the upper leg 414 and about the heel portion 458 of the prosthetic foot 454. In addition, the cord 486 can be attached by being looped about a pin or hook extending from the pylon 452 or the upper leg 414 and clamped between the prosthetic foot 454 and the lower leg 424. In general, the cord 486 can be attached by being clamped or otherwise fastened to at least two of the upper end of the pylon 452, the lower end 453 of the pylon 452, the upper leg 414, the lower leg 424, and the prosthetic foot 454.

As shown in FIGS. 11 and 13, the prosthetic foot 454 preferably includes two or more toe sections 518 (e.g., a lateral section and a medial section) formed in the toe portion 456 for a split-keel prosthetic foot, although any other type of prosthetic foot 454 can instead be used as desired. One of the toe sections 518 (i.e., the medial section) can have a smaller width than the other and can be positioned medially with respect to a longitudinal axis 445 of the pylon 452. The medially-positioned toe section can be used to simulate a preference toward where the amputee's big toe would be located on the amputee's left or right side. In addition, the pylon 452 and/or the prosthetic ankle 412 can include two or more sections (e.g., a lateral section and a medial section) that move independently of one another to allow for torsional and lateral movements of the prosthetic walking system 450.

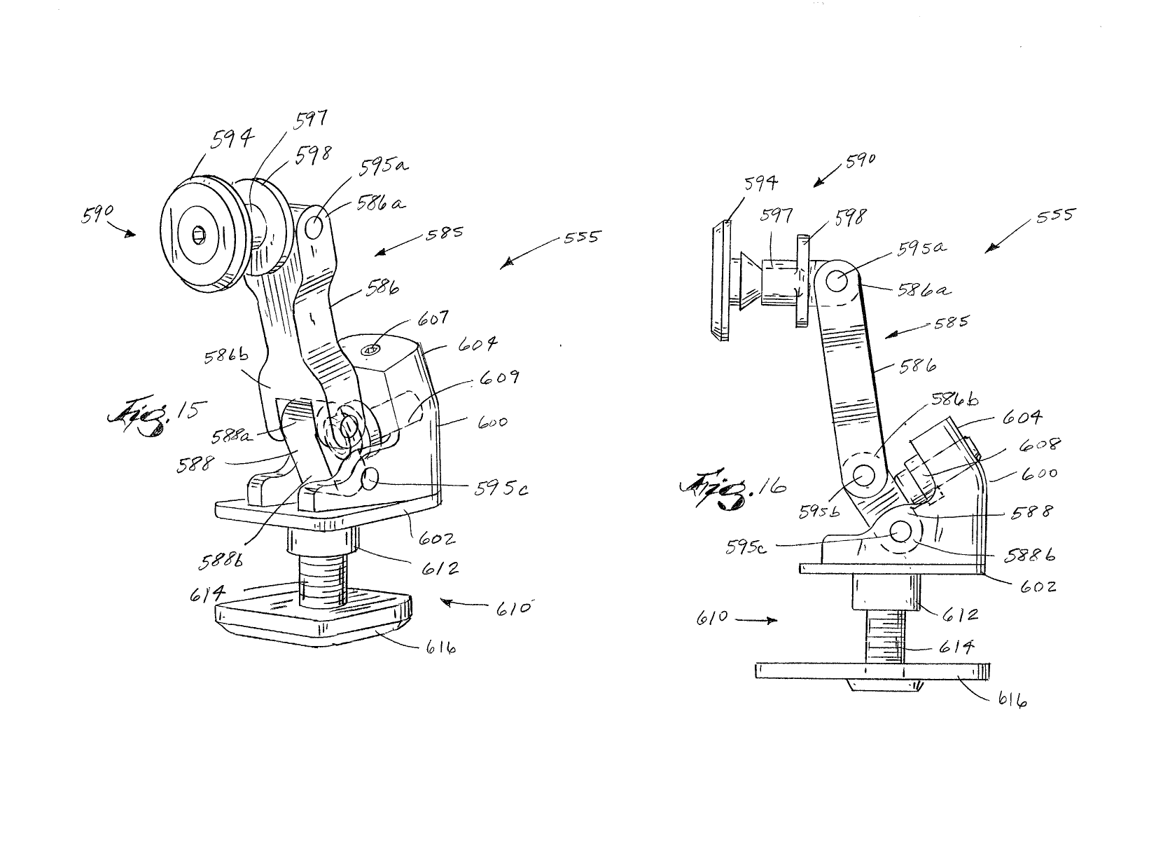

FIGS. 14-19 illustrate a prosthetic walking system 550 which is an alternative embodiment of the prosthetic walking systems 50, 150, 250, 350, and 450 described above. Elements and features of the prosthetic walking system 650 illustrated in FIGS. 14-19 having a form, structure, or function similar to that found in the prosthetic walking system of FIGS. 3-13 are given corresponding reference numbers in the 500-600 series. The prosthetic walking system 550 preferably includes a pylon 552 integrally connected to a prosthetic ankle assembly 510, and can take any of the forms described above with reference to the preferred embodiments of FIGS. 9A-13. Alternatively, the pylon 552 and prosthetic ankle 512 can take any of the forms and can be connected in any of the manners described above with reference to the preferred embodiments of FIGS. 3-8. The prosthetic ankle assembly 510 illustrated in FIGS. 14-19 is comprised of a prosthetic ankle 512 and a link assembly 555, as will be described below. A lower end 553 of the pylon 552 is preferably integrally connected to an upper leg 514 of the prosthetic ankle 512. The upper leg 514 is preferably integrally connected to an interconnecting portion 532, which is preferably integrally connected to a lower leg 524. Preferably, the interconnecting portion 432 flexes at a medial/lateral axis 534.

As shown in FIGS. 14-19, the link assembly 555 can be embodied by an adjustable link assembly 585 (which is an alternative embodiment of the limit strap assembly 485 illustrated in FIGS. 11-13). The adjustable link assembly 585 preferably includes a first link 586, a second link 588, and a heel 600. Preferably, the first link 586 has a first portion or end 586a that is coupled to an upper post assembly 590. The upper post assembly 590 includes an upper bolt 594 threaded through a hole 593 in the pylon 552 and into a threaded barrel 592. Preferably, the threaded barrel 592 includes a cylindrical aperture 596 through which a first pivot pin 595a is positioned. The first pivot pin 595a preferably permits the first end 586a of the first link 586 to rotate about a longitudinal axis of the first pivot pin 595a, which is preferably parallel or substantially parallel to the medial/lateral axis 534 about which the prosthetic ankle 512 flexes. However, the first end 586a of the first link 586 can also be rigidly positioned within the cylindrical aperture 596.

The first link 586 preferably has a second portion or end 586b that is coupled to a first portion or end 588a of the second link 588 by a second pivot pin 595b. The second end 586b of the first link 586 preferably rotates about a longitudinal axis of the second pivot pin 595b, which is also preferably parallel to the medial/lateral axis 534. A second portion or end 588b of the second link 588 is preferably coupled to the heel binding 600. Preferably, the second end 588b is coupled to the heel 600 via a third pivot pin 595c positioned within holes 601 in a bottom portion 602 of the heel 600. A longitudinal axis of the third pivot pin 595c is also preferably parallel to the medial/lateral axis 534.

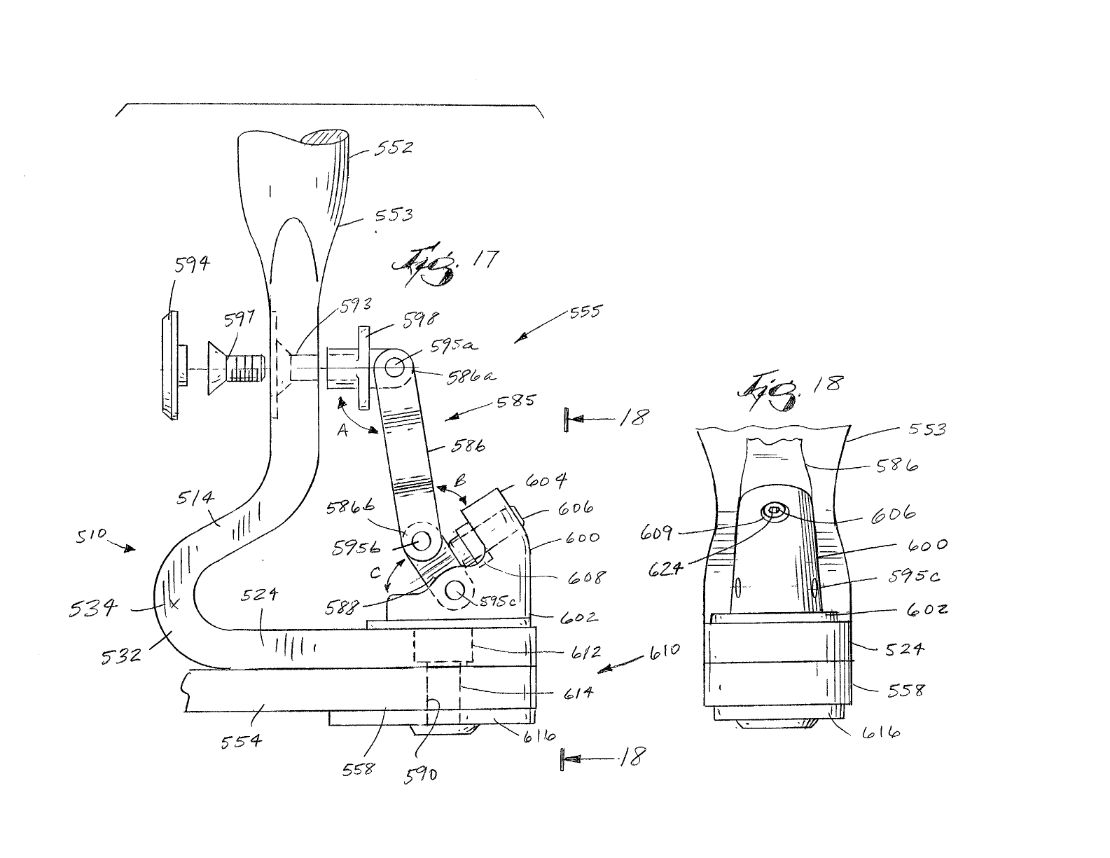

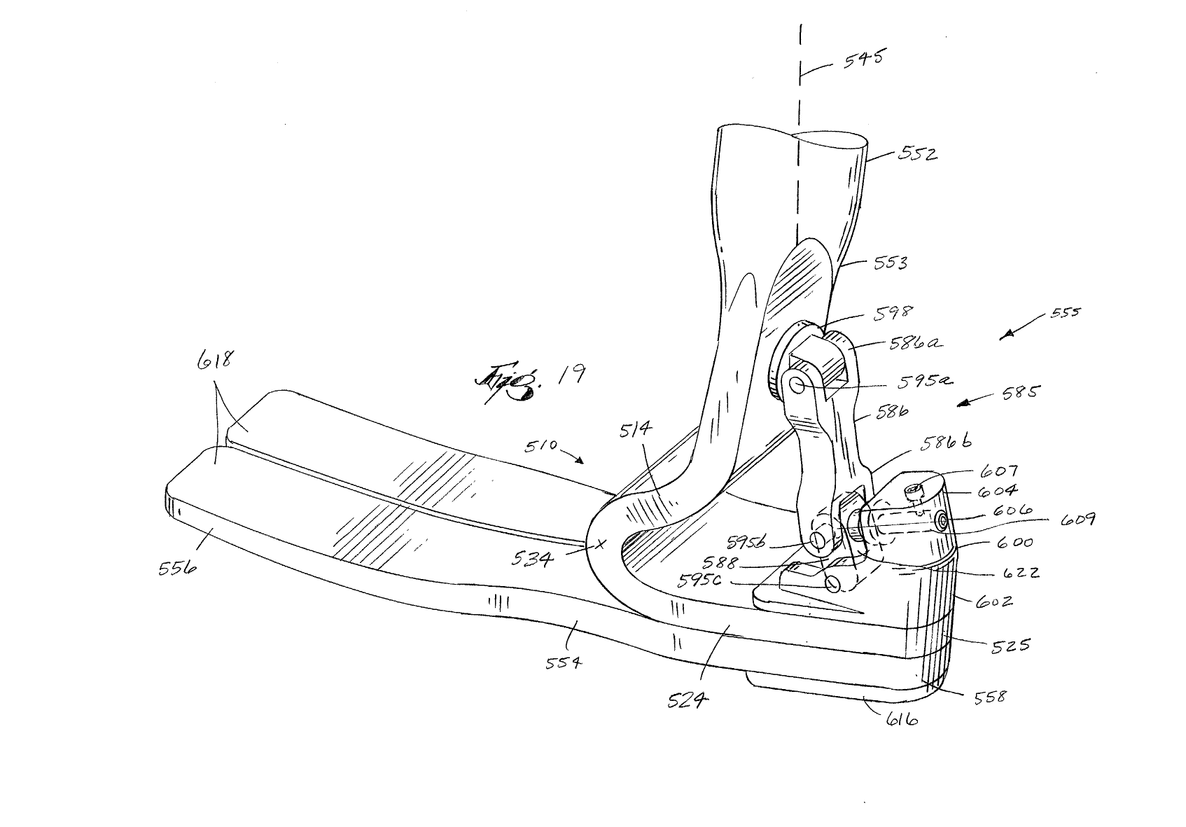

As best shown in FIG. 17, the heel 600 is preferably coupled to the prosthetic foot 554 through a bolt assembly 610. FIGS. 15 and 16 illustrate the adjustable link assembly 585 and bolt assembly 610 removed from the prosthetic walking system 550. The bolt assembly 610 preferably includes a shank 612, a threaded shaft 614 depending from the shank 612, and an attachment plate 616. As shown in FIG. 17, the shank 612 and the threaded shaft 614 are preferably positioned within a receiving hole 590 which passes through both the lower leg 524 of the prosthetic ankle 512 and the heel portion 558 of prosthetic foot 554. The lower leg 524 and the heel portion 558 can thereby be secured between the bottom portion 602 of the heel 600 and the attachment plate 616.

The operation of the prosthetic walking system 550 is best described with reference to FIG. 17. At heel-strike when the amputee's weight is placed on the prosthetic ankle 512, the upper leg 514 and the lower leg 524 flex toward one another about the medial/lateral axis 534. The first link 586 preferably rotates clockwise as viewed in FIG. 17 (i.e., in the anterior direction) about the first pivot pin 595a through an arc A (as designated in FIG. 17) toward the lower portion 553 of the pylon 552. At the same time, the first end 588a of the second link 588 rotates about the second pivot pin 595b through an arc B (also as designated in FIG. 17) as the second link 588 rotates counter-clockwise as viewed in FIG. 17. In addition, the second end 588b of the second link 588 rotates downwardly about the third pivot pin 595c through an arc C (also as designated in FIG. 17). As a result, the first end 586a of the first link 586 and the second end 588b of the second link 588 each rotate in the posterior direction toward one another as the upper leg 514 and the lower leg 524 flex toward one another.

At toe-off when the amputee's weight is taken off of the prosthetic ankle 512, the motion of the adjustable link assembly 585 is reversed. Specifically, the first end 586a of the first link 586 and the second end 588b of the second link 588 separate from one another until the link assembly 585 restrains the upper leg 514 and the lower leg 524 from flexing apart from one another any farther.

The maximum displacement between the first end 586a of the first link 586 and the second end 588b of the second link 588, and thus, the maximum displacement between the upper leg 514 and the lower leg 524, is preferably adjustable. As best shown in FIG. 14, an adjustment screw 606 can be provided to adjust the maximum displacement between the upper leg 514 and the lower leg 524. The adjustment screw 606 preferably includes a threaded shaft 618 that can be advanced into and out of a hole 605 in the upper portion 604 of the heel binding 600. A bumper 608 is preferably attached to the an anterior head 620 of the adjustment screw 606 with a collar 609. The bumper 608 is preferably positioned between the anterior head 620 of the adjustment screw 606 and a posterior face 622 of the second linkage 588. The bumper 608 engages the posterior face 622 of the second link 588 so that the second link 588 is prevented from moving in the posterior direction beyond the position of the bumper 608 and the anterior head 620 of the adjustment screw 606. Also, in other embodiments, the bumper 608 can be attached directly to the second link 588 (with or without the collar 609), rather than to the anterior head 620 of the adjustment screw 606.

The bumper 608 (and preferably the collar 609) can be constructed of a compressible material with a high coefficient of friction, such as rubber or a rubber-like material. Other bumper materials include without limitation urethane, nylon, UHMW Polyethylene or one of its commercial versions Lenite® (Westlake Plastics Company) or Tivar 1000® (Poly Hi Solidue, Inc.), plastic, Teflon® (E.I. du Pont de Nemours and Company), and the like. Alternatively, the bumper 608 and the collar 609 can be omitted so that the anterior head 620 of the adjustment screw 606 itself engages the posterior face 622 of the second link 588.

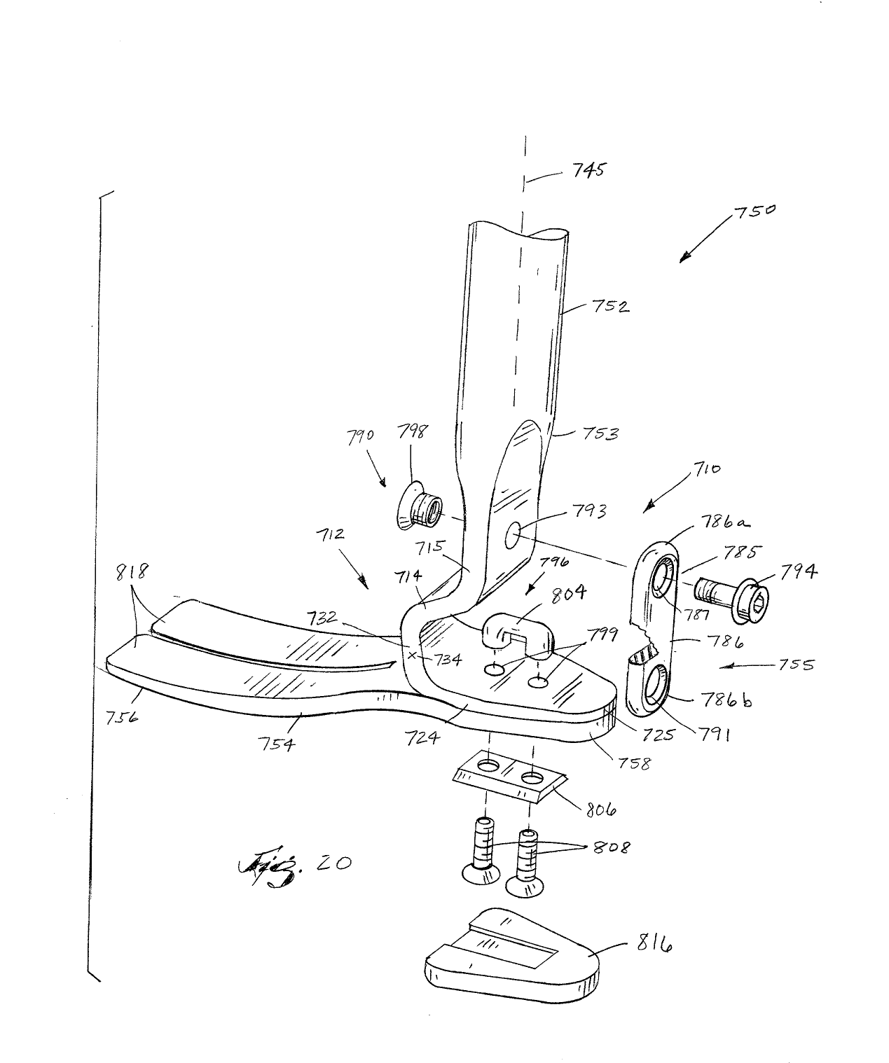

As best shown in FIGS. 18 and 19, a rear access hole 609 is preferably located in the heel 600 in order to access and adjust the adjustment screw 606. The adjustment screw 606 is preferably configured with a faceted recess 624 which can be engaged with an appropriate tool, such as an Allen wrench, in order to rotate the adjustment screw 606.

Any number of other devices can also be used to adjust the position of the bumper 608 or to adjust the positions of the first link 586 and/or the second link 588. For example, a bolt received within a hole in the heel 600 with a bolt head positioned adjacent the circumference of an access hole in the heel 600 can be used to adjust the bumper 608, the first link 586, and/or the second link 588. Also, a pin received within a hole in the heel 600 and held in different positions therein by a setscrew resting on one of a series of flats or recesses along the length of the pin can be used to adjust the bumper 608, the first link 586, and/or the second link 588. In addition, a cotter pin (i.e., a pin having a series of holes along its length through which a wire, clip, or pin can be inserted) can be received within a hole in the heel 600 and used to adjust the bumper 608, the first link 586, and/or the second link 588. Moreover, the bumper 608 can be slidably mounted upon a rail, track, or beam on the heel 600, and can be retained in two or more positions thereon by conventional fasteners, clips, clamps, etc. In general, any conventional adjustment element or assembly that can be used to retain the bumper 608 in various positions with respect to the first link 586 and/or the second link 588 can instead be used and falls within the spirit and scope of the present invention.

Preferably, when the adjustment screw 606 is tightened (i.e., advanced out of the hole 605 in the anterior direction), the second link 588 rotates about the longitudinal axis of the second pivot pin 595b, and the first end 588a of the second link 588 moves toward the interconnecting portion 532 and toward the lower leg 524. Thus, tightening the adjustment screw 606 preferably reduces the distance between the first end 586a of the first link 586 and the second end 588b of the second link 588. Tightening the adjustment screw 606 also increases the tension that the adjustable link assembly 585 applies between the upper leg 514 and the lower leg 524. This increase in tension can reduce the maximum displacement between the upper leg 514 and the lower leg 524 as the amputee's weight W is taken off the pylon 552 at toe-off.

Preferably, when the adjustment screw 606 is loosened (i.e., advanced into the hole 605 in the posterior direction), the second link 588 rotates about the longitudinal axis of the second pivot pin 595b, and the first end 588a of the second link 588 moves away from the interconnecting portion 532 and away from the lower leg 524. Thus, loosening the adjustment screw 606 preferably increases the distance between the first end 586a of the first link 586 and the second end 588b of the second link 588 by permitting these ends 586a, 588b to spread apart under the force of the interconnecting portion 532. Loosening the adjustment screw 606 also preferably reduces the tension that the adjustable link assembly 585 applies between the upper leg 514 and the lower leg 524. This decrease in tension increases the maximum displacement between the upper leg 514 and the lower leg 524 as the amputee's weight W is taken off the pylon 552 at toe-off.

In order to fix the adjustment screw 606 at a selected position, a stop screw 607 (as best shown in FIG. 14) is preferably positioned within a hole 611 in the upper portion 604 of the heel 600. The stop screw 607 preferably engages the one or more flats on the threaded shaft 618 of the adjustment screw 606 to prevent movement of the adjustment screw 606. Alternatively, the position of the adjustment screw 606 can be fixed by using a screw locking material or patch on the adjustment screw 606, by using a self-locking screw, or by using a screw and threaded hole having self-locking threads. Other adjustment devices can use other well-known elements to fix the adjustment screw 606 in a desired position.

As shown in FIG. 19, the prosthetic foot 554 preferably includes two or more toe sections 618 (e.g., a lateral section and a medial section) formed in the toe portion 556 for a split-keel prosthetic foot, although any type of prosthetic foot can be used in conjunction with the prosthetic ankle 512 described above and illustrated in FIGS. 14-19. One of the toe sections 618 (i.e., the medial section) can have a smaller width and/or can be positioned medially with respect to a longitudinal axis 545 of the pylon 552. A medially-positioned toe section can be used to simulate a preference toward where the amputee's big toe would be located on the amputee's left or right side. In addition, the pylon 552 and/or the prosthetic ankle 512 can include two or more sections (e.g., a lateral section and a medial section) that move independently of one another to allow for torsional and lateral movements of the prosthetic walking system 550.

In some embodiments, rather than including the first link 586 and the second link 588 as described above, the adjustable link assembly 585 can include a hydraulic or pneumatic cylinder, an air spring or any other adjustable or non-adjustable spring (including without limitation torsion, leaf, and helical springs), coupled between the upper post assembly 590, the pylon 552, or the upper leg 514 and the bolt assembly 610, the lower leg 524, or the heel portion 558 of the prosthetic foot 554. In some embodiments, the pressure within the hydraulic or pneumatic cylinder or provided by the air spring can be adjusted in order to at least partially define the maximum displacement between the upper leg 514 and the lower leg 524 of the prosthetic ankle 512.

The link assembly 585 shown in FIGS. 14-19 can take a number of alternative forms, each one of which employs at least two links coupled together and then coupled to the pylon 552 and/or the upper leg 514 and to the lower leg 524 and/or the prosthetic foot 554. Each link can have any shape (even disc-shaped), and need not be connected at an identifiable “end,” so long as the link is connected to another link and connected to the pylon 552, the upper leg 514, the lower leg 524, and/or the prosthetic foot 554 to provide motion similar to that shown in FIGS. 14-19. The links can be connected to the pylon 552, the upper leg 514, the lower leg 524, and/or the prosthetic foot 554 in a number of different manners, and the use of the upper post assembly 590 and the heel 600 for the connections is only one alternative. For example, each link can be coupled to a hook or U-shaped element attached to or integrally formed with the pylon 552, the upper leg 514, the lower leg 524, and/or the prosthetic foot 554. The links preferably pivot about these elements to provide movement similar to that shown in FIGS. 14-19. Moreover, the maximum displacement of any alternative link assembly can be limited in a number of different manners. The adjustment screw 606 having the bumper 608 in FIGS. 14-19 can be positioned to limit any of the links, and in this regard can be positioned to contact other parts of the links (if desired) to perform the displacement-limiting function. The device need not necessarily be adjustable, and only needs to limit motion of one of the links in any manner to perform its intended displacement-limiting function. Any device or element capable of doing performing the displacement-limiting function falls within the spirit and scope of the present invention.

FIG. 20 illustrates a prosthetic walking system 750 which is an alternative embodiment of the prosthetic walking systems 50, 150, 250, 350, 450, and 550 described above. Elements and features of the prosthetic walking system 750 illustrated in FIG. 20 having a form, structure, or function similar to that found in the prosthetic walking system of FIGS. 3-19 are given corresponding reference numbers in the 700-800 series. The prosthetic walking system 750 preferably includes a pylon 752 integrally connected to a prosthetic ankle assembly 710 and can take any of the forms described above with reference to the preferred embodiments of FIGS. 9A-13. Alternatively, the pylon 752 and prosthetic ankle 712 can take any of the forms and can be connected in any of the manners described above with reference to the preferred embodiments of FIGS. 3-8. The prosthetic ankle assembly 710 illustrated in FIG. 20 is comprised of a prosthetic ankle 712 and a link assembly 755, as will be described below. A lower end 753 of the pylon 752 is preferably integrally connected to an upper leg 714 of the prosthetic ankle 712. The upper leg 714 is preferably integrally connected to an interconnecting portion 732, which is preferably integrally connected to a lower leg 724. The interconnecting portion 732 preferably flexes at a medial/lateral axis 734.

FIG. 20 illustrates a limit belt assembly 785 which is an embodiment of a link assembly similar to the link assemblies 55, 455, and 555 described above. Although only shown and described with respect to the prosthetic walking system 750, the limit belt assembly 785 is also suitable for use with the prosthetic walking systems 50, 150, 250, 350, 450, and 550 and their alternative embodiments described above, and particularly with the prosthetic walking systems 250, 350, 450, and 550 which have an integral construction. The limit belt assembly 785 includes a link comprised of a resilient belt 786 that limits the flexion of the upper leg 714 and the lower leg 724 apart from one another.

The belt 786 can be constructed of any resilient, flexible, abrasion-resistant material that is strong enough to limit the biasing force of the interconnecting region 732. Also, the belt 786 can be constructed of a combination of materials in order to achieve the desired characteristics. For example, materials such as nylon, rubber, polypropylene, polyester, cotton, Nomex®, or Kevlar® (both manufactured by E.I. du Pont de Nemours and Company) can be used alone or in combination to construct the belt 786. Also, the belt 786 can have any degree of flexibility-from not elongating as the amputee's weight shifts between heel-strike and toe-off to easily elongating as the amputee's weight shifts between heel-strike and toe-off. For example, in order to achieve similar results, the belt 786 could be shorter and elongate more easily or the belt 786 could be longer and elongate less easily as the amputee's weight shifts between heel-strike and toe-off.

Preferably, the belt 786 includes an upper aperture 787 and a lower aperture 791. Preferably, an upper post assembly 790 is used to couple the belt 786 to the upper leg 714 and/or the pylon 752 through the upper aperture 787, and a lower post assembly 796 is used to couple the belt 786 to the lower leg 724 and/or the heel portion 758 of the foot 754 through the lower aperture 791. In this regard, the upper post assembly 790 is preferably attached at a posterior end 715 of the upper leg 714 or at the lower end 753 of the pylon 752. The upper post assembly 790 preferably includes an upper bolt 794 positioned through a hole 793 in the lower end 753 of the pylon 752 or in the upper leg 714 and coupled to a threaded-receiving barrel 798.

The lower post assembly 796 preferably includes a lower post 804, a screw plate 806, screws 808, and an attachment plate 816. The lower post 804 is preferably positioned through the lower aperture 791 of the belt 786. The lower post assembly 796 is preferably attached to the heel portion 758 of the prosthetic foot 754 and to the lower leg 724 via the screws 808. The screws 808 are preferably positioned through the screw plate 806 and holes 799, which pass through both the lower leg 724 of the prosthetic ankle 712 and the heel portion 758 of the prosthetic foot 754. The screws 808 are preferably received within mating threaded apertures (not shown) in the lower post 804 in order to secure the lower post 804 to the lower leg 724 and prosthetic foot 754. The attachment plate 816 can be used to cover the heads of the screws 808 and the screw plate 806 and to protect the screws 808 and screw plate 806 from wear.

The belt 786 can be connected to the pylon 752 and/or the upper leg 714 and to the lower leg 724 and/or the prosthetic foot 754 in any number of other conventional manners, including those described above with reference to the manners in which the limit strap 55, the cord 486, and the first link 586 and the second link 588 are connected. Each of these alternative manners of connection falls within the spirit and scope of the present invention. For example, the upper bolt 794 can simply be tightened into the hole 793 without using threaded-receiving barrel 798. Also, the lower leg 724 can have an upwardly-turned flange with a hole through which another bolt passes to connect through the lower aperture 791 in a manner similar to the upper aperture 787. In another embodiments, hooks can be integrally formed or attached to the pylon 752, the upper leg 714, the lower leg 724, and/or the prosthetic foot 754 and the hooks can be looped into the upper aperture 791 and the lower aperture 787.

The prosthetic foot 754 preferably includes two or more toe sections 818 (e.g., a lateral section and a medial section) formed in the toe portion 756 for a split-keel prosthetic foot, although any type of prosthetic foot can be used in conjunction with the prosthetic ankle 712 described above and illustrated in FIG. 20. One of the toe sections 818 (i.e., the medial section) may have a smaller width and/or can be positioned medially with respect to a longitudinal axis 745 of the pylon 752. The medially-positioned toe section can be used to simulate a preference toward where the amputee's big toe would be located on the amputee's left or right side. Although a split-keep prosthetic foot is shown and described with respect to the prosthetic walking systems 450, 550, and 750, a split-keel prosthetic foot is equally suitable for use in any of the other prosthetic walking systems 50, 150, 250, and 350 described herein. In addition, the pylon and/or the prosthetic ankle can include two or more sections (e.g., a lateral section and a medial section) that move independently of one another to allow for torsional and lateral movements of any of the prosthetic walking systems 50, 150, 250, 250, 450, 550, and 750.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

We claim:

1. A prosthetic walking system for attachment to an amputee, the prosthetic walking system comprising:
a pylon having an upper end for attachment to the amputee and a lower end;
a prosthetic foot;
an prosthetic ankle coupled between the pylon and the prosthetic foot, the prosthetic ankle having
an upper leg coupled to the lower end of the pylon;
a lower leg coupled to the prosthetic foot; and
an interconnecting portion located between the upper leg and the lower leg; and
at least one link coupled to at least one of the lower end of the pylon and the upper leg, the at least one link also coupled to at least one of the lower leg and the prosthetic foot, the at least one link at least partially defining a maximum displacement between the upper leg and the lower leg.

2. The prosthetic walking system of claim 1, wherein:
the upper leg has an anterior portion;
the lower leg has an anterior portion; and
the interconnecting portion is located between the anterior portion of the upper leg and the anterior portion of the lower leg.

3. The prosthetic walking system of claim 2, wherein:
the upper leg has a posterior portion;
the lower leg has a posterior portion; and
the at least one link is coupled between the posterior portion of the upper leg and the posterior portion of the lower leg.

4. The prosthetic walking system of claim 1, wherein the upper leg and the lower leg of the prosthetic ankle are substantially straight and the interconnecting portion of the prosthetic ankle is substantially arcuate.

5. The prosthetic walking system of claim 1, wherein the upper leg, the lower leg, and the interconnecting portion of the prosthetic ankle are each substantially arcuate.

6. The prosthetic walking system of claim 1, wherein the pylon and the prosthetic ankle are an integral unit.

7. The prosthetic walking system of claim 1, wherein the at least one link is a resilient belt having
an upper portion coupled to at least one of the pylon and the upper leg of the prosthetic ankle; and
a lower portion coupled to at least one of the lower leg of the prosthetic ankle and the prosthetic foot.

8. The prosthetic walking system of claim 7, wherein the resilient belt is a cord extending at least twice between at least one of the pylon and the upper leg of the prosthetic ankle and at least one of the lower leg of the prosthetic ankle and the prosthetic foot.

9. The prosthetic walking system of claim 1, wherein the at least one link is a strap having
a top portion coupled between the pylon and the upper leg of the prosthetic ankle;
a bottom portion coupled between the prosthetic ankle and the prosthetic foot; and
an intermediate portion located between the top portion and the bottom portion, a length of the intermediate portion at least partially defining the maximum displacement between the upper leg and the lower leg.

10. The prosthetic walking system of claim 1, wherein the at least one link includes
a first link having a first portion and a second portion, the first portion of the first link being coupled to at least one of the pylon and the upper leg of the prosthetic ankle;
a second link having a first portion and a second portion, the first portion of the second link being coupled to the second portion of the first link; and
a heel having a top portion and a bottom portion, the top portion of the heel being coupled to the second portion of the second link, the bottom portion of the heel being coupled to at least one of the lower leg of the prosthetic ankle and the prosthetic foot.

11. The prosthetic walking system of claim 10, further comprising an adjustment screw coupled to at least one of the first link and the second link and to the heel, wherein the adjustment screw is adjustable to vary the maximum displacement between the upper leg and the lower leg of the prosthetic ankle.

12. The prosthetic walking system of claim 1, wherein the at least one link includes at least one of a hydraulic cylinder and a pneumatic cylinder coupled to at least one of the pylon and the upper leg of the prosthetic ankle and to at least one of the lower leg of the prosthetic ankle and the prosthetic foot.

13. The prosthetic walking system of claim 1, wherein the upper leg of the prosthetic ankle has a first length and the lower leg of the prosthetic ankle has a second length greater than the first length.

14. The prosthetic walking system of claim 1, wherein at least a portion of the prosthetic ankle is flexible.

15. The prosthetic walking system of claim 14, wherein the interconnecting portion is flexible.

16. The prosthetic walking system of claim 1, wherein at least a portion of the prosthetic ankle flexes before the pylon flexes when a load is placed on the prosthetic walking system.

17. The prosthetic walking system of claim 1, wherein:
the prosthetic ankle has a cross-sectional shape having a first moment of inertia and the pylon has a cross-sectional shape having a second moment of inertia; and
the first moment of inertia is less than the second moment of inertia so that the prosthetic ankle flexes before the pylon flexes when a load is placed on the prosthetic walking system.

18. The prosthetic walking system of claim 1, wherein the pylon has a first width and a portion of the prosthetic ankle has a second width smaller than the first width so that the prosthetic ankle flexes before the pylon flexes when a load is placed on the prosthetic walking system.

19. The prosthetic walking system of claim 18, wherein the portion of the prosthetic ankle having the second width is positioned asymmetrically with respect to a longitudinal axis of the pylon.

20. The prosthetic walking system of claim 1, wherein the pylon has a substantially circular cross-sectional shape and the prosthetic ankle has a substantially rectangular cross-sectional shape.

21. The prosthetic walking system of claim 1, wherein:
the pylon is constructed of a first material;
the prosthetic ankle is constructed of a different second material; and
the second material is more compliant than the first material so that the prosthetic ankle flexes before the pylon flexes when a load is placed on the prosthetic walking system.

22. The prosthetic walking system of claim 21, wherein the first material is carbon-fiber composite and the second material is fiberglass.

23. The prosthetic walking system of claim 1, wherein at least one of the pylon, the prosthetic ankle, and the prosthetic foot includes a lateral section independently movable with respect to a medial section.

24. The prosthetic walking system of claim 23, wherein the prosthetic foot includes a toe portion and the toe portion includes the lateral section and the medial section.

25. The prosthetic walking system of claim 24, wherein:
the lateral section has a first width; and
the medial section has a second width smaller than the first width.

26. A method of adjusting a prosthetic walking system according to an amputee's gait, the method comprising:
attaching a prosthetic walking system to the amputee, the prosthetic walking system including a pylon, a prosthetic foot, and a prosthetic ankle coupled between the pylon and the prosthetic foot, the prosthetic ankle having an upper leg, a lower leg, and an interconnecting portion located between the upper leg and the lower leg;
providing at least one link coupled between at least one of the pylon and the upper leg and at least one of the lower leg and the prosthetic foot;
limiting the maximum displacement between the upper leg and the lower leg with the at least one link; and
adjusting the at least one link to change the maximum displacement between the upper leg and the lower leg.

27. The method of claim 26, further comprising rotating an adjustment screw to adjust the at least one link.

28. The method of claim 26, further comprising changing a pressure in at least one of a hydraulic cylinder and a pneumatic cylinder to adjust the at least one link.

29. A prosthetic walking system for attachment to an amputee, the prosthetic walking system comprising:
a pylon having an upper end for attachment to the amputee and a lower end;
a prosthetic foot having a heel portion;
an prosthetic ankle coupled between the pylon and the prosthetic foot, the prosthetic ankle having
an upper leg coupled to the lower end of the pylon, the upper leg having an anterior portion;
a lower leg coupled to the heel portion, the lower leg having an anterior portion; and
an interconnecting portion located between the anterior portion of the upper leg and the anterior portion of the lower leg; and
a link assembly at least partially defining a maximum displacement between the upper leg and the lower leg, the link assembly including
a first link having a first portion and a second portion, the first portion of the first link coupled to the pylon;
a second link having a first portion and a second portion, the first portion of the second link coupled to the second portion of the first link; and
a heel having a first portion and a second portion, the first portion of the heel coupled to the second portion of the second link, the second portion of the heel coupled to the heel portion of the prosthetic foot.

30. The prosthetic walking system of claim 29, wherein:
the second portion of the first link is rotatably coupled to the first portion of the second link;
the second portion of the second link is rotatably coupled to the first portion of the heel; and
the first link and the second link are pivotably responsive to flexure of the prosthetic ankle.

31. The prosthetic walking system of claim 29, wherein the upper leg, the lower leg, and the interconnecting portion of the prosthetic ankle are each substantially arcuate.

32. The prosthetic walking system of claim 29, wherein the upper leg of the prosthetic ankle has a first length and the lower leg of the prosthetic ankle has a second length greater than the first length.

33. The prosthetic walking system of claim 29, wherein at least a portion of the prosthetic ankle is flexible.

34. The prosthetic walking system of claim 33, wherein the interconnecting portion is flexible.

35. The prosthetic walking system of claim 29, wherein at least a portion of the prosthetic ankle flexes before the pylon flexes when a load is placed on the prosthetic walking system.

36. The prosthetic walking system of claim 29, wherein:
the prosthetic ankle has a cross-sectional shape having a first moment of inertia and the pylon has a cross-sectional shape having a second moment of inertia; and
the first moment of inertia is less than the second moment of inertia so that the prosthetic ankle flexes before the pylon flexes when a load is placed on the prosthetic walking system.

37. The prosthetic walking system of claim 29, wherein the pylon has a first width and a portion of the prosthetic ankle has a second width smaller than the first width so that the prosthetic ankle flexes before the pylon flexes when a load is placed on the prosthetic walking system.

38. The prosthetic walking system of claim 29, wherein the pylon has a substantially circular cross-sectional shape and the prosthetic ankle has a substantially rectangular cross-sectional shape.

39. The prosthetic walking system of claim 29, wherein:
the pylon is constructed of a first material;
the prosthetic ankle is constructed of a different second material; and
the second material is more compliant than the first material so that the prosthetic ankle flexes before the pylon flexes when a load is placed on the prosthetic walking system.

40. The prosthetic walking system of claim 39, wherein the first material is carbon-fiber composite and the second material is fiberglass.

41. A prosthetic walking system for attachment to an amputee, the prosthetic walking system comprising:
a pylon having an upper end for attachment to the amputee and a lower end;
a prosthetic ankle integral with the pylon, the prosthetic ankle including
an upper leg having an anterior portion and a posterior portion, the posterior portion being integral with the lower end of the pylon;
a lower leg having an anterior portion and a posterior portion; and
an interconnecting portion located between the anterior portion of the upper leg and the anterior portion of the lower leg; and
a prosthetic foot coupled to at least one of the anterior portion and the posterior portion of the lower leg of the prosthetic ankle.

42. The prosthetic walking system of claim 41, wherein the upper leg and the lower leg of the prosthetic ankle are substantially straight and the interconnecting portion of the prosthetic ankle is substantially arcuate.

43. The prosthetic walking system of claim 41, wherein the upper leg, the lower leg, and the interconnecting portion of the prosthetic ankle are each substantially arcuate.

44. The prosthetic walking system of claim 41, further comprising at least one link coupled between at least one of the lower end of the pylon and the upper leg and at least one of the lower leg and the prosthetic foot, the at least one link at least partially defining a maximum displacement between the upper leg and the lower leg.

45. The prosthetic walking system of claim 44, wherein the at least one link is a resilient belt having an upper portion coupled to at least one of the pylon and the upper leg of the prosthetic ankle; and
a lower portion coupled to at least one of the lower leg of the prosthetic ankle and the prosthetic foot.

46. The prosthetic walking system of claim 45, wherein the resilient belt is a cord extending at least twice between at least one of the pylon and the upper leg of the prosthetic ankle and at least one of the lower leg of the prosthetic ankle and the prosthetic foot.

47. The prosthetic walking system of claim 44, wherein the at least one link is a strap having
a top portion coupled between the pylon and the upper leg of the prosthetic ankle;
a bottom portion coupled between the prosthetic ankle and the prosthetic foot; and
an intermediate portion located between the top portion and the bottom portion, a length of the intermediate portion at least partially defining the maximum displacement between the upper leg and the lower leg.

48. The prosthetic walking system of claim 44, wherein the at least one link includes
a first link having a first portion and a second portion, the first portion of the first link being coupled to at least one of the pylon and the upper leg of the prosthetic ankle;
a second link having a first portion and a second portion, the first portion of the second link being coupled to the second portion of the first link; and
a heel having a top portion and a bottom portion, the top portion of the heel being coupled to the second portion of the second link, the bottom portion of the heel being coupled to at least one of the lower leg of the prosthetic ankle and the prosthetic foot.

49. The prosthetic walking system of claim 48, further comprising an adjustment screw coupled between at least one of the first link and the second link and the heel, wherein the adjustment screw is adjustable in order to vary the maximum displacement between the upper leg and the lower leg of the prosthetic ankle.

50. The prosthetic walking system of claim 44, wherein the at least one link includes at least one of a hydraulic cylinder and a pneumatic cylinder coupled to at least one of the pylon and the upper leg of the prosthetic ankle and to at least one of the lower leg of the prosthetic ankle and the prosthetic foot.

51. The prosthetic walking system of claim 41, wherein the upper leg of the prosthetic ankle has a first length and the lower leg of the prosthetic ankle has a second length greater than the first length.

52. The prosthetic walking system of claim 41, wherein at least a portion of the prosthetic ankle is flexible.

53. The prosthetic walking system of claim 52, wherein the interconnecting portion is flexible.

54. The prosthetic walking system of claim 41, wherein at least a portion of the prosthetic ankle flexes before the pylon flexes when a load is placed on the prosthetic walking system.

55. The prosthetic walking system of claim 41, wherein:
the prosthetic ankle has a cross-sectional shape having a first moment of inertia and the pylon has a cross-sectional shape having a second moment of inertia; and
the first moment of inertia is less than the second moment of inertia so that the prosthetic ankle flexes before the pylon flexes when a load is placed on the prosthetic walking system.

56. The prosthetic walking system of claim 41, wherein the pylon has a first width and a portion of the prosthetic ankle has a second width smaller than the first width so that the prosthetic ankle flexes before the pylon flexes when a load is placed on the prosthetic walking system.

57. The prosthetic walking system of claim 56, wherein the portion of the prosthetic ankle having the second width is positioned asymmetrically with respect to a longitudinal axis of the pylon.

58. The prosthetic walking system of claim 41, wherein the pylon has a substantially circular cross-sectional shape and the prosthetic ankle has a substantially rectangular cross-sectional shape.

59. The prosthetic walking system of claim 41, wherein:
the pylon is constructed of a first material;
the prosthetic ankle is constructed of a different second material; and
the second material is more compliant than the first material so that the prosthetic ankle flexes before the pylon flexes when a load is placed on the prosthetic walking system.

60. The prosthetic walking system of claim 59, wherein the first material is carbon-fiber composite and the second material is fiberglass.

61. The prosthetic walking system of claim 41, wherein at least one of the pylon, the prosthetic ankle, and the prosthetic foot includes a lateral section independently movable with respect to a medial section.

62. The prosthetic walking system of claim 61, wherein the prosthetic foot includes a toe portion and the toe portion includes the lateral section and the medial section.

63. The prosthetic walking system of claim 62, wherein:
the lateral section has a first width; and
the medial section has a second width smaller than the first width.

64. A prosthetic walking system for attachment to an amputee, the prosthetic walking system comprising:
a pylon having an upper end for attachment to the amputee and a lower end;
a prosthetic foot; and
an prosthetic ankle coupled between the pylon and the prosthetic foot, the prosthetic ankle having
an upper leg coupled to the lower end of the pylon;
a lower leg coupled to the prosthetic foot;
an interconnecting portion located between the upper leg and the lower leg; and
a weakened portion defined within at least one of the upper leg and the interconnecting portion, the weakened portion being less resistant to bending than the pylon so that the prosthetic walking system flexes at the weakened portion when a load is placed on the prosthetic walking system by the amputee.

65. The prosthetic walking system of claim 64, wherein the pylon has a first width and the weakened portion has a second width smaller than the first width.

66. The prosthetic walking system of claim 65, wherein the weakened portion having the second width is positioned asymmetrically with respect to a longitudinal axis of the pylon.

67. The prosthetic walking system of claim 64, wherein the pylon has a first cross-sectional area and the weakened portion has a second cross-sectional area smaller than the first cross-sectional area.

68. The prosthetic walking system of claim 64, wherein:
the weakened portion has a cross-sectional shape having a first moment of inertia and the pylon has a cross-sectional shape having a second moment of inertia; and
the first moment of inertia is less than the second moment of inertia so that the prosthetic ankle flexes before the pylon flexes when a load is placed on the prosthetic walking system.

69. The prosthetic walking system of claim 64, wherein the pylon has a substantially circular cross-sectional shape and the prosthetic ankle has a substantially rectangular cross-sectional shape.

70. The prosthetic walking system of claim 64, wherein:
the pylon is constructed of a first material;
the weakened portion is constructed of a different second material; and
the second material is more compliant than the first material so that the weakened portion flexes before the pylon flexes when a load is placed on the prosthetic walking system.

71. The prosthetic walking system of claim 70, wherein the first material is carbon-fiber composite and the second material is fiberglass.

72. The prosthetic walking system of claim 64, wherein the upper leg and the lower leg of the prosthetic ankle are substantially straight and the interconnecting portion of the prosthetic ankle is substantially arcuate.

73. The prosthetic walking system of claim 64, wherein the upper leg, the lower leg, and the interconnecting portion of the prosthetic ankle are each substantially arcuate.

74. The prosthetic walking system of claim 64, further comprising at least one link coupled between at least one of the lower end of the pylon and the upper leg and at least one of the lower leg and the prosthetic foot, the at least one link at least partially defining a maximum displacement between the upper leg and the lower leg.

75. The prosthetic walking system of claim 74, wherein the at least one link is a resilient belt having
an upper portion coupled to at least one of the pylon and the upper leg of the prosthetic ankle; and
a lower portion coupled to at least one of the lower leg of the prosthetic ankle and the prosthetic foot.

76. The prosthetic walking system of claim 75, wherein the resilient belt is a cord extending at least twice between at least one of the pylon and the upper leg of the prosthetic ankle and at least one of the lower leg of the prosthetic ankle and the prosthetic foot.

77. The prosthetic walking system of claim 74, wherein the at least one link is a strap having
a top portion coupled between the pylon and the upper leg of the prosthetic ankle;
a bottom portion coupled between the prosthetic ankle and the prosthetic foot; and
an intermediate portion coupled between the top portion and the bottom portion, a length of the intermediate portion defining the maximum displacement between the upper leg and the lower leg.

78. The prosthetic walking system of claim 74, wherein the at least one link includes
a first link having a first portion and a second portion, the first portion of the first link being coupled to at least one of the pylon and the upper leg of the prosthetic ankle;
a second link having a first portion and a second portion, the first portion of the second link being coupled to the second portion of the first link; and
a heel having a top portion and a bottom portion, the top portion of the heel being coupled to the second portion of the second link, the bottom portion of the heel being coupled to at least one of the lower leg of the prosthetic ankle and the prosthetic foot.

79. The prosthetic walking system of claim 78, further comprising an adjustment screw coupled to at least one of the first link and the second link and to the heel, wherein the adjustment screw is adjustable to vary the maximum displacement between the upper leg and the lower leg of the prosthetic ankle.

80. The prosthetic walking system of claim 74, wherein the at least one link includes at least one of a hydraulic cylinder and a pneumatic cylinder coupled to at least one of the pylon and the upper leg of the prosthetic ankle and to at least one of the lower leg of the prosthetic ankle and the prosthetic foot.

81. The prosthetic walking system of claim 64, wherein the upper leg of the prosthetic ankle has a first length and the lower leg of the prosthetic ankle has a second length greater than the first length.

82. The prosthetic walking system of claim 64, wherein at least a portion of the prosthetic ankle is flexible.

83. The prosthetic walking system of claim 82, wherein the interconnecting portion is flexible.

84. The prosthetic walking system of claim 64, wherein at least a portion of the prosthetic ankle flexes before the pylon flexes when a load is placed on the prosthetic walking system.

85. The prosthetic walking system of claim 64, wherein at least one of the pylon, the prosthetic ankle, and the prosthetic foot includes a lateral section independently movable with respect to a medial section.

86. The prosthetic walking system of claim 85, wherein the prosthetic foot includes a toe portion and the toe portion includes the lateral section and the medial section.

87. The prosthetic walking system of claim 86, wherein:
the lateral section has a first width; and
the medial section has a second width smaller than the first width.

88. A prosthetic walking system for attachment to an amputee, the prosthetic walking system comprising:
a pylon having an upper end for attachment to the amputee and a lower end;
a prosthetic foot; and
a prosthetic ankle coupled between the pylon and the prosthetic foot, the prosthetic ankle having
an upper leg coupled to the lower end of the pylon by a first connection;
a lower leg coupled to the prosthetic foot by a second connection; and
an interconnecting portion located between the upper leg and the lower leg;
at least one of (a) the first connection being adjustable so that the lower end of the pylon can be coupled to the upper leg in at least two positions; and (b) the second connection being adjustable so that the prosthetic foot can be coupled to the lower leg in at least two positions.

89. The prosthetic walking system of claim 88, wherein:
the upper leg has an aperture adapted to receive the lower end of the pylon; and
the aperture has a first portion adapted to receive the pylon in a first position and a second portion adapted to receive the pylon in a second position.

90. The prosthetic walking system of claim 88, wherein the pylon has a substantially circular cross-sectional area and the prosthetic ankle has a substantially rectangular cross-sectional area.

91. The prosthetic walking system of claim 88, wherein at least a portion of the prosthetic ankle is flexible.

92. The prosthetic walking system of claim 91, wherein the interconnecting portion is flexible.

93. The prosthetic walking system of claim 88, wherein at least a portion of the prosthetic ankle flexes before the pylon flexes when a load is placed on the prosthetic walking system.

94. The prosthetic walking system of claim 88, wherein the pylon has a first width and a portion of the prosthetic ankle has a second width smaller than the first width so that the prosthetic ankle flexes before the pylon flexes when a load is placed on the prosthetic walking system.

95. The prosthetic walking system of claim 94, wherein the portion of the prosthetic ankle having the second width is positioned asymmetrically with respect to a longitudinal axis of the pylon.

96. The prosthetic walking system of claim 88, wherein:
the prosthetic ankle has a cross-sectional shape having a first moment of inertia and the pylon has a cross-sectional shape having a second moment of inertia; and
the first moment of inertia is less than the second moment of inertia so that the prosthetic ankle flexes before the pylon flexes when a load is placed on the prosthetic walking system.

97. The prosthetic walking system of claim 88, wherein:
the pylon is constructed of a first material;
the prosthetic ankle is constructed of a different second material; and
the second material is more compliant than the first material so that the prosthetic ankle flexes before the pylon flexes when a load is placed on the prosthetic walking system.

98. The prosthetic walking system of claim 97, wherein the first material is carbon-fiber composite and the second material is fiberglass.

99. The prosthetic walking system of claim 88, wherein the upper leg and the lower leg of the prosthetic ankle are substantially straight and the interconnecting portion of the prosthetic ankle is substantially arcuate.

100. The prosthetic walking system of claim 88, wherein the upper leg, the lower leg, and the interconnecting portion of the prosthetic ankle are each substantially arcuate.

101. The prosthetic walking system of claim 88, further comprising at least one link coupled between at least one of the lower end of the pylon and the upper leg and at least one of the lower leg and the prosthetic foot, the at least one link at least partially defining a maximum displacement between the upper leg and the lower leg.

102. The prosthetic walking system of claim 101, wherein the at least one link is a resilient belt having
an upper portion coupled to at least one of the pylon and the upper leg of the prosthetic ankle; and
a lower portion coupled to at least one of the lower leg of the prosthetic ankle and the prosthetic foot.

103. The prosthetic walking system of claim 102, wherein the resilient belt is a cord extending at least twice between at least one of the pylon and the upper leg of the prosthetic ankle and at least one of the lower leg of the prosthetic ankle and the prosthetic foot.

104. The prosthetic walking system of claim 101, wherein the at least one link is a strap having
a top portion coupled between the pylon and the upper leg of the prosthetic ankle;
a bottom portion coupled between the prosthetic ankle and the prosthetic foot; and
an intermediate portion coupled between the top portion and the bottom portion, a length of the intermediate portion defining the maximum displacement between the upper leg and the lower leg.

105. The prosthetic walking system of claim 101, wherein the at least one link includes
a first link having a first portion and a second portion, the first portion of the first link being coupled to at least one of the pylon and the upper leg of the prosthetic ankle;
a second link having a first portion and a second portion, the first portion of the second link being coupled to the second portion of the first link; and
a heel having a top portion and a bottom portion, the top portion of the heel being coupled to the second portion of the second link, the bottom portion of the heel being coupled to at least one of the lower leg of the prosthetic ankle and the prosthetic foot.

106. The prosthetic walking system of claim 105, further comprising an adjustment screw coupled to at least one of the first link and the second link and to the heel, wherein the adjustment screw is adjustable to vary the maximum displacement between the upper leg and the lower leg of the prosthetic ankle.

107. The prosthetic walking system of claim 101, wherein the at least one link includes at least one of a hydraulic cylinder and a pneumatic cylinder coupled to at least one of the pylon and the upper leg of the prosthetic ankle and to at least one of the lower leg of the prosthetic ankle and the prosthetic foot.

108. The prosthetic walking system of claim 88, wherein the upper leg of the prosthetic ankle has a first length and the lower leg of the prosthetic ankle has a second length greater than the first length.

109. The prosthetic walking system of claim 88, wherein at least one of the pylon, the prosthetic ankle, and the prosthetic foot includes a lateral section independently movable with respect to a medial section.

110. The prosthetic walking system of claim 109, wherein the prosthetic foot includes a toe portion and the toe portion includes the lateral section and the medial section.

111. The prosthetic walking system of claim 110, wherein:
the lateral section has a first width; and
the medial section has a second width smaller than the first width.
